(19) United States
(12) Patent Application Publication
(10) Pub. No.: US 2026/0041818 A1
(43) Pub. Date: Feb. 12, 2026

(54) BIOENGINEERED INTERNAL ANAL SPHINCTER CONSTRUCTS AND METHODS OF USE THEREOF

(71) Applicant: CELLF-BIO, INC., Chantilly, VA (US)

(72) Inventors: Khalil Nakhle Bitar, Chantilly, VA (US); Prabhash Dadhich, Henrico, VA (US)

(21) Appl. No.: 19/100,368

(22) PCT Filed: Aug. 1, 2023
(86) PCT No.: PCT/US2023/071455

§ 371 Date: Jan. 31, 2025

Related U.S. Application Data
(60) Provisional application No. 63/394,549, filed on Aug. 2, 2022.

(51) Int. Cl. A61L 27/44; A61L 27/22; A61L 27/24; A61L 27/36; A61L 27/52; A61L 27/58; C12N 5/077; C12N 5/0797

(52) U.S. Cl. CPC A61L 27/44; A61L 27/227; A61L 27/24; A61L 27/3604; A61L 27/3687; A61L 27/52; A61L 27/58; C12N 5/0623; C12N 5/0661; A61L 2430/22; C12N 2533/52; C12N 2533/54

(57) ABSTRACT

Disclosed herein are internal anal sphincter constructs, and methods for generating and/or using such constructs. Also provided herein are methods for using internal anal sphincter construct(s) for treating fecal incontinence in a subject. The method of generating an innervated internal anal sphincter construct, provided herein, comprises using smooth muscle cells (SMCs) from an anorectum tissue cell biopsy and neural progenitor cells (NPCs) from an intestinal tissue cell biopsy from the subject.

### CLAIM OF PRIORITY

This application claims the benefit of U.S. Provisional Application Ser. No. 63/394,549, filed on Aug. 2, 2022. The entire contents of the foregoing are incorporated herein by reference.

### FEDERALLY SPONSORED RESEARCH OR DEVELOPMENT

This invention was made with Government support under Grant No. DK105593 awarded by the National Institutes of Health. The Government has certain rights in the invention.

### TECHNICAL FIELD

Described herein are methods of bioengineering anal sphincter constructs, the constructs themselves, and methods of use thereof.

### BACKGROUND

Fecal incontinence (FI), the involuntary soiling of various amounts of liquid and solid stool, is often devastating from a social, psychological, and hygiene perspective. Men and women suffer from FI equally with a range of 2-6% in people aged 20-30 years. The prevalence increases to over 15% in people older than 70 years. FI may result from an isolated or combined loss of smooth muscle function (IAS), skeletal muscle function (EAS), anorectal sensory mechanisms, or neural control. Treatment of FI is initially conservative with attempts to bulk the stool, adjust or stop drugs that cause either diarrhea or constipation, and biofeedback. While these conservative measures may improve the severity of FI, additional therapies are needed for total symptom management. Injection of various biomaterials to augment the internal anal sphincter has also been attempted, but results have been variable and of limited duration when effective. In the past, surgical attempts with anterior/posterior surgical repair have been tried such as graciloplasty and sacral nerve stimulation, but long-term results have been disappointing. Hence, there is a critical need for new therapies to address FI, particularly for patients who have severe passive incontinence and low IAS pressure.

### SUMMARY

Provided herein are methods for generating an innervated internal anal sphincter construct. The methods include isolating smooth muscle cells (SMCs) from an anorectum tissue cell biopsy from a subject; isolating neural progenitor cells (NPCs) from an intestinal tissue cell biopsy from the subject; suspending the NPCs in collagen and/or laminin hydrogel, and mixing the NPCs with the SMCs to provide a cell mix; seeding the cell mix as a single-layer hydrogel matrix on a mold and allowing gelation of the cell mix around a central post; contacting the single-layer hydrogel matrix with a neural differentiation media; and allowing maturation of the single-layer hydrogel matrix and differentiation of the NPCs, thereby forming the innervated internal anal sphincter construct having directionally oriented SMCs.

Also provided herein are methods for treating fecal incontinence (FI) in a subject in need thereof. The methods include isolating smooth muscle cells (SMCs) from an anorectum tissue cell biopsy obtained from the subject; isolating neural progenitor cells (NPCs) from an intestinal tissue cell biopsy obtained from the subject; suspending the NPCs in collagen and/or laminin hydrogel, and mixing the NPCs with the SMCs to provide a cell mix; seeding the cell mix as a single-layer hydrogel matrix on a mold and allowing gelation of the cell mix around a central post; contacting the single-layer hydrogel matrix with a neural differentiation media; allowing maturation of the single-layer hydrogel matrix and differentiation of the NPCs, thereby forming an innervated internal anal sphincter construct having directionally oriented SMCs; and implanting the innervated internal anal sphincter construct to the subject, wherein the implantation is through a circumferential dissection around the anorectum of the subject.

In some embodiments, the single-layer hydrogel matrix comprises NPCs and SMCs in a ratio of about 1:1 to about 1:1000 (e.g., about 1:1 to about 1:10, about 1:10 to about 1:100, or about 1:100 to about 1:1000). In some embodiments, the single-layer hydrogel matrix comprises NPCs and SMCs in a ratio of about 1:2 to about 1:5. In some embodiments, the single-layer hydrogel matrix comprises NPCs and SMCs in a ratio of about 1:2.5. In some embodiments, the single-layer hydrogel matrix comprises laminin and collagen in a ratio of about 1:5 to about 1:1000 (e.g., about 1:5 to about 1:10, about 1:5 to about 1:50, about 1:5 to about 1:100, or about 1:5 to about 1:500).

In some embodiments, about 0.5×10⁶ to about 2500×10⁶ SMCs (e.g., about 0.5×10⁶ to about 25×10⁶ SMCs, about 25×10⁶ to about 250×10⁶ SMCs, or about 250×10⁶ to about 2500×10⁶ SMCs) are isolated from the anorectum tissue cell biopsy from the subject.

In some embodiments, the methods further include obtaining the anorectum tissue cell biopsy from the subject. In some embodiments, the anorectum tissue cell biopsy is obtained from internal anal sphincter tissue of the subject.

In some embodiments, the anorectum tissue cell biopsy weighs at least about 50 mg, e.g., about 200 mg.

In some embodiments, about 0.2×10⁶ to about 1000×10⁶ NPCs (e.g., about 0.2×10⁶ to about 10×10⁶ NPCs, about 10×10⁶ to about 100×10⁶ NPCs, or about 100×10⁶ to about 1000×10⁶ NPCs) are isolated from the intestinal tissue cell biopsy from the subject. In some embodiments, the methods further include obtaining the intestinal tissue cell biopsy from the subject.

In some embodiments, the intestinal tissue cell biopsy is obtained from jejunal small intestine tissue of the subject. In some embodiments, the intestinal tissue cell biopsy is obtained laparoscopically. In some embodiments, the intestinal tissue cell biopsy weighs at least about 50 mg, e.g., about 200 mg.

In some embodiments, the single-layer hydrogel matrix is matured to form the innervated internal anal sphincter construct in about 3-12 days (e.g., about 3-5 days, 5-7 days, 7-9 days, 9-11 days, or 10-12 days).

In some embodiments, the innervated internal anal sphincter construct comprises less than 20% bovine collagen.

In some embodiments, the innervated internal anal sphincter construct comprises a central open lumen, wherein the central open lumen has a diameter of about 20-25 mm.

In some embodiments, multiple (e.g., 6-8) innervated internal anal sphincter constructs are generated, and a portion thereof are implanted in the subject. In some embodiments, four innervated internal anal sphincter constructs are implanted in the subject.

In some embodiments, the subject is diagnosed with fecal incontinence, shows one or more symptoms of fecal incontinence, and/or is at a risk of developing fecal incontinence.

In some embodiments, the subject is a human.

As used herein, the terms “about” and “approximately” are used as equivalents. Any numerals used in this disclosure with or without about/approximately are meant to cover any normal fluctuations appreciated by one of ordinary skill in the relevant art. The term “approximately” or “about” refers to a range of values that fall within 10% or less in either direction (greater than or less than) of the stated reference value unless otherwise stated or otherwise evident from the context (except where such number would exceed 100% of a possible value). Unless otherwise defined, all technical and scientific terms used herein have the same meaning as commonly understood by one of ordinary skill in the art to which this disclosure belongs. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIG. 1 is a schematic representation of the process of isolation and engineering the sphincters. The overall process starts with biopsy collection and cell isolation (Step 1), bioengineering of the Sphincters (Step 2), and implantation of the BioSphincters (Step 3).

FIG. 2 is a schematic representation of a process flow chart describing the details of manufacturing and release.

FIG. 3 is a schematic representation of an exemplary method of generating a BioSphincter. A 20 mm diameter silicone post is mounted in the center of a silicone-coated 60 mm diameter plate. A cell mix comprising smooth muscle cells (SMCs) and neural progenitor cells (NPCs) is seeded as a single layer gel matrix around the post and is followed by the formation of a BioSphincter around the post that matures over 12 days.

FIG. 4 shows a BioSphincter that has a ring structure with a central lumen. The internal diameter of the BioSphincter is 20 mm. The surface area of the BioSphincters averaged 140.90±0.85 mm² and the volume averaged 153.87±0.62 mm³. The thickness of the BioSphincter averaged 1.92±0.01 mm. The height of the BioSphincter averaged 2.44±0.01 mm. Values are means±SEM; N=3.

FIG. 5 shows the final product transport vessel. (A) The set-up of the vessel consists of a (1) baffle, (2) a stainless-steel rod with two grooves one on each end for the (3) O-Rings to sit on. (B) Final assembly of the set-up. (C) Following the placement of 4 BioSphincters around the rod, the O-rings are mounted in place, and the rod is placed in the 50-mL tube. The baffle is placed on the top part of the rod. The tube is then filled with transport media and the tube is capped.

FIG. 6 shows qPCR analysis of human collagen synthesized by SMCs during the bioengineering process (upper panel) and compares cell synthesized collagen and bovine collagen (lower panel).

FIGS. 7A-7B show characterization of cells. (A) Isolated smooth muscle cells proliferated and acquired a normal spindle-like morphology. (B) Isolated neural progenitor cells.

FIGS. 8A-8B shows glucose consumption and lactate production in the BioSphincters. Media was collected at media changes starting on day 6 and until the final product harvest (day 12). Media was analyzed for (A) glucose consumption and (B) lactate production. Data showed that glucose consumption and lactate production were increased in the BioSphincters over the days of the study (n=3; mean±SEM).

FIGS. 9A-9C shows the physiological functionality of the BioSphincters. The BioSphincters were tested for the functionality of the smooth muscle and neural components.

FIG. 10 shows the viability of the BioSphincters. Viability of the BioSphincters was followed from day 1 up to day 12 post-bioengineering using MTT assay. The viability of the cells in the BioSphincters decreased between day 1 and day 12 by only 3%, which was not statistically significant.

FIG. 11 shows the physiological functionality of BioSphincters stored at different temperatures for up to 48 hours. BioSphincters responded similarly to stimulation of both smooth muscle and neural components. (n=3)

FIG. 12 shows the viability of BioSphincters at different temperatures. BioSphincters showed no significant change in viability when stored at different temperatures for up to 48 hours when compared to control (n=3 per condition).

### DETAILED DESCRIPTION

Fecal incontinence is the inability to control bowel movements, wherein stool (feces) unexpectedly leaks from the rectum. Symptoms of fecal incontinence (particularly related to the loss of control) can range from mild and occasional to frequent and severe. Causes of fecal incontinence can include, for example, trauma, such as giving birth or could be associated with aging. A non-functioning internal anal sphincter could be from muscle damage, making it difficult to hold stool back, or could be from nerve damage, making it difficult to sense stool in the rectum or some combination thereof. An injury to the perineum may result in complete or partial destruction of the anal sphincter and the distal rectum. Such damage could potentially result in persistent incontinence or the need for a permanent colostomy. Anorectal continence is maintained by interplay between the enteric nervous system, smooth muscle internal anal sphincter, the striated external anal sphincter, and puborectalis muscles. The internal anal sphincter contributes >70% of the basal tension required to maintain continence. Damage to the integrity of the anorectum can result in fecal incontinence. Additionally, loss of internal anal sphincter integrity and function can be a result of aging, anorectal surgery, and/or medical comorbidity. Again, these causes alone or collectively can lead to the treatment of fecal incontinence. The resulting psychological stress, social stigma, decreased self-esteem and productivity can be overwhelming. Overall, treatment strategies for fecal incontinence remain limited.

Current treatments for fecal incontinence are not optimal. Treatments include biofeedback, sacral nerve stimulation, myoblast injections, bulking agents, and artificial anal sphincter implantations. These technologies focus either on reinstatement of the striated muscle of the external anal sphincter or mechanical closure using artificial devices, with little focus on the reinstatement or preservation of terminal gut function. Terminal gut function requires coordinated contraction and relaxation of the smooth muscle of the rectum and internal anal sphincter mediated by the enteric nervous system.

There is a need for internal anal sphincter constructs that can be used for restoration of internal anal sphincter function of a subject—in particular, subjects for whom other standard therapies have failed. The present methods and compositions can be used to restore internal anal sphincter function, including both muscle and nerve function. Methods of the present disclosure include using cellular components, including smooth muscle and intrinsic neural components for engineering, regenerating, and/or reinstating healthy tissues and/or treating fecal incontinence. In some embodiments, constructs, as provided herein, are capable of reestablishing function to the internal anal sphincter of a subject in need thereof. In some embodiments, the present disclosure provides methods of forming such constructs.

Among other things, the present disclosure provides internal anal sphincter (IAS) constructs and methods for generating and/or using such constructs. Also provided herein are methods for treating fecal incontinence (FI) using internal anal sphincter construct(s). The IAS constructs comprise two types of cells: (1) smooth muscle cells (SMC) isolated from the IAS and (2) enteric neural progenitor cells (NPC) from the small intestine (jejunum).

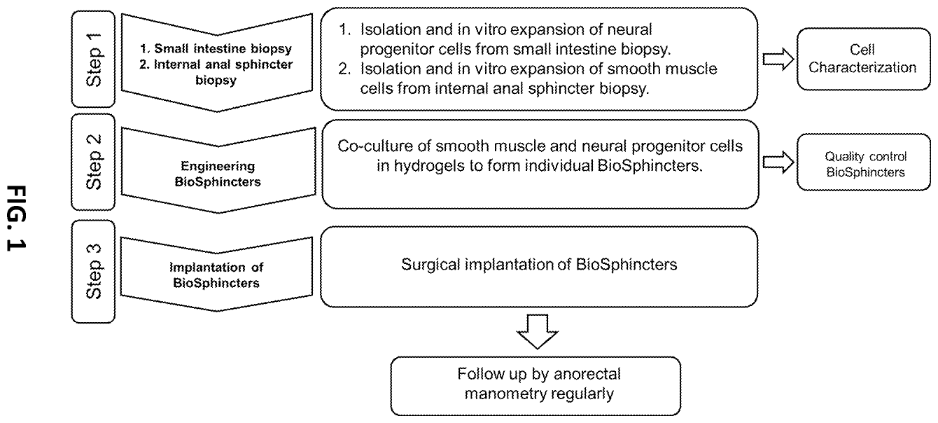

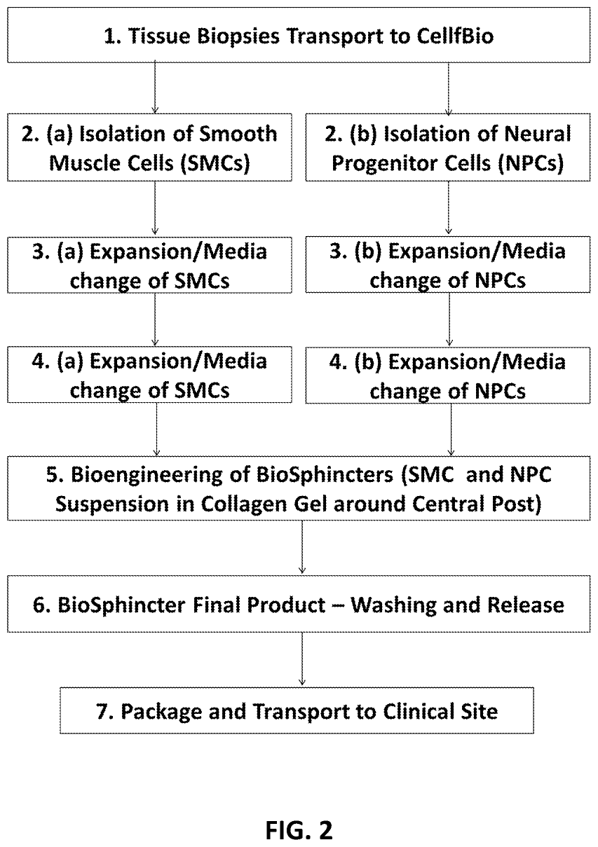

An exemplary process of isolation and engineering of the internal anal sphincter construct(s) is summarized in FIG. 1. Details of manufacturing and release of internal anal sphincter construct(s) is represented in a process flow chart in FIG. 2. FIG. 3 illustrates the engineering process of a BioSphincter using IAS smooth muscle cells and neural progenitor cells. Generally speaking, the overall process starts with biopsy collection and cell isolation, followed by bioengineering of the Sphincters. Both cell types are co-cultured in a hydrogel and allowed to form intrinsically innervated, concentrically aligned, circular IAS constructs (referred to as BioSphincter). For example, neural progenitor cells isolated from a patient are collected, suspended in collagen/laminin gel, and mixed with smooth muscle cells (preferably from the same patient). This mixture is allowed to gel around a central post of a mold, e.g., for at least 30-180 minutes (e.g., at least 30-60 minutes, 60-90 minutes, 90-120 minutes, 120-150 minutes, or 150-180 minutes; such as, at least 30 minutes, 60 minutes, 90 minutes, 120 minutes, 150 minutes, or 180 minutes) at about 20-40° C. (e.g., about 20-25° C., 25-30° C., 30-35° C., or 35-40° C.; such as, about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.) and about 1-10% CO₂ (e.g., about 1% CO₂, 2% CO₂, 3% CO₂, 4% CO₂, 5% CO₂, 6% CO₂, 7% CO₂, 8% CO₂, 9% CO₂, or 10% CO₂). Following gelation, differentiation media is added to the plate. Subsequently, the SMCs contract the gels into a ring-like structure around the post to form one IAS construct. The IAS construct is cultured, e.g., for 10-14 days, e.g., about 12 days, with media changes as needed. Further exemplary details are provided below.

### Cells

Disclosed herein are methods for generating bioengineered internal anal sphincters (IAS) constructs by fabricating them using neural progenitor cells (NPCs) and smooth muscle cells (SMCs), preferably autologous NPCs and SMCs. The SMCs are preferably obtained from internal anal sphincter of a subject (e.g., a mammal, such as a human), while the NPCs are preferably obtained from intestine (e.g., small intestine, such as jejunum) of a subject (e.g., a mammal, such as a human). For example, SMCs can be isolated from an anorectum tissue cell biopsy of a subject, and NPCs can be isolated from an intestinal tissue cell biopsy of a subject. Accordingly, also disclosed here are methods of preparing and taking an anorectum tissue cell biopsy and/or an intestinal tissue cell biopsy from a subject. Such biopsies (e.g., anorectum tissue cell biopsy and/or intestinal tissue cell biopsy) can be obtained by laparoscopic procedures.

For generation of bioengineered IAS construct(s) described herein, about 0.5×10⁶ to about 2500×10⁶ SMCs, such as, about 0.5×10⁶ to about 25×10⁶ SMCs (such as, about 1×10⁶ to about 25×10⁶ SMCs, about 2.5×10⁶ to about 25×10⁶ SMCs, about 5×10⁶ to about 25×10⁶ SMCs, about 7.5×10⁶ to about 25×10⁶ SMCs, about 10×10⁶ to about 25×10⁶ SMCs, about 12.5×10⁶ to about 25×10⁶ SMCs, about 15×10⁶ to about 25×10⁶ SMCs, about 17.5×10⁶ to about 25×10⁶ SMCs, or about 20×10⁶ to about 25×10⁶ SMCs (e.g., 0.5×10⁶ SMCs, 0.75×10⁶ SMCs, 1×10⁶ SMCs, 1.25×10⁶ SMCs, 1.5×10⁶ SMCs, 1.75×10⁶ SMCs, 2×10⁶ SMCs, 2.25×10⁶ SMCs, 2.5×10⁶ SMCs, 2.75×10⁶ SMCs, 3×10⁶ SMCs, 3.25×10⁶ SMCs, 3.5×10⁶ SMCs, 3.75×10⁶ SMCs, 4×10⁶ SMCs, 4.25×10⁶ SMCs, 4.5×10⁶ SMCs, 4.75×10⁶ SMCs, 5×10⁶ SMCs, 5.5×10⁶ SMCs, 6×10⁶ SMCs, 6.5×10⁶ SMCs, 7×10⁶ SMCs, 7.5×10⁶ SMCs, 8×10⁶ SMCs, 8.5×10⁶ SMCs, 9×10⁶ SMCs, 9.5×10⁶ SMCs, 10×10⁶ SMCs, 10.5×10⁶ SMCs, 11×10⁶ SMCs, 11.5×10⁶ SMCs, 12×10⁶ SMCs, 12.5×10⁶ SMCs, 13×10⁶ SMCs, 13.5×10⁶ SMCs, 14×10⁶ SMCs, 14.5×10⁶ SMCs, 15×10⁶ SMCs, 15.5×10⁶ SMCs, 16×10⁶ SMCs, 16.5×10⁶ SMCs, 17×10⁶ SMCs, 17.5×10⁶ SMCs, 18×10⁶ SMCs, 18.5×10⁶ SMCs, 19×10⁶ SMCs, 19.5×10⁶ SMCs, 20×10⁶ SMCs, 20.5×10⁶ SMCs, 21×10⁶ SMCs, 21.5×10⁶ SMCs, 22×10⁶ SMCs, 22.5×10⁶ SMCs, 23×10⁶ SMCs, 23.5×10⁶ SMCs, 24×10⁶ SMCs, 24.5×10⁶ SMCs, or 25×10⁶ SMCs)), about 25×10⁶ to about 250×10⁶ SMCs (such as, about 50×10⁶ to about 250×10⁶ SMCs, about 75×10⁶ to about 250×10⁶ SMCs, about 100×10⁶ to about 250×10⁶ SMCs, about 125×10⁶ to about 250×10⁶ SMCs, about 150×10⁶ to about 250×10⁶ SMCs, about 175×10⁶ to about 250×10⁶ SMCs, or about 200×10⁶ to about 250×10⁶ SMCs (e.g., about 25×10⁶ SMCs, 27.5×10⁶ SMCs, 30×10⁶ SMCs, 32.5×10⁶ SMCs, 35×10⁶ SMCs, 37.5×10⁶ SMCs, 40×10⁶ SMCs, 42.5×10⁶ SMCs, 45×10⁶ SMCs, 47.5×10⁶ SMCs, 50×10⁶ SMCs, 55×10⁶ SMCs, 60×10⁶ SMCs, 65×10⁶ SMCs, 70×10⁶ SMCs, 75×10⁶ SMCs, 80×10⁶ SMCs, 85×10⁶ SMCs, 90×10⁶ SMCs, 95×10⁶ SMCs, 100×10⁶ SMCs, 105×10⁶ SMCs, 110×10⁶ SMCs, 115×10⁶ SMCs, 120×10⁶ SMCs, 125×10⁶ SMCs, 130×10⁶ SMCs, 135×10⁶ SMCs, 140×10⁶ SMCs, 145×10⁶ SMCs, 150×10⁶ SMCs, 155×10⁶ SMCs, 160×10⁶ SMCs, 165×10⁶ SMCs, 170×10⁶ SMCs, 175×10⁶ SMCs, 180×10⁶ SMCs, 185×10⁶ SMCs, 190×10⁶ SMCs, 195×10⁶ SMCs, 200×10⁶ SMCs, 205×10⁶ SMCs, 210×10⁶ SMCs, 215×10⁶ SMCs, 220×10⁶ SMCs, 225×10⁶ SMCs, 230×10⁶ SMCs, 235×10⁶ SMCs, 240×10⁶ SMCs, 245×10⁶ SMCs, or 250×10⁶ SMCs)), or about 250×10⁶ to about 2500×10⁶ SMCs (such as, about 300×10⁶ to about 2500×10⁶ SMCs, about 350×10⁶ to about 2500×10⁶ SMCs, about 400×10⁶ to about 2500×10⁶ SMCs, about 450×10⁶ to about 2500×10⁶ SMCs, about 500×10⁶ to about 2500×10⁶ SMCs, about 550×10⁶ to about 2500×10⁶ SMCs, about 600×10⁶ to about 2500×10⁶ SMCs, about 650×10⁶ to about 2500×10⁶ SMCs, about 700×10⁶ to about 2500×10⁶ SMCs, about 750×10⁶ to about 2500×10⁶ SMCs, about 800×10⁶ to about 2500×10⁶ SMCs, about 850×10⁶ to about 2500×10⁶ SMCs, about 900×10⁶ to about 2500×10⁶ SMCs, about 950×10⁶ to about 2500×10⁶ SMCs, about 1000×10⁶ to about 2500×10⁶ SMCs, about 1250×10⁶ to about 2500×10⁶ SMCs, about 1500×10⁶ to about 2500×10⁶ SMCs, about 1750×10⁶ to about 2500×10⁶ SMCs, about 2000×10⁶ to about 2500×10⁶ SMCs, or about 2250×10⁶ to about 2500×10⁶ SMCs (e.g., about 300×10⁶ SMCs, about 350×10⁶ SMCs, about 400×10⁶ SMCs, about 450×10⁶ SMCs, about 500×10⁶ SMCs, about 550×10⁶ SMCs, about 600×10⁶ SMCs, about 650×10⁶ SMCs, about 700×10⁶ SMCs, about 750×10⁶ SMCs, about 800×10⁶ SMCs, about 850×10⁶ SMCs, about 900×10⁶ SMCs, about 950×10⁶ SMCs, about 1000×10⁶ SMCs, about 1250×10⁶ SMCs, about 1500×10⁶ SMCs, about 1750×10⁶ SMCs, about 2000×10⁶ SMCs, about 2250×10⁶ SMCs, or about 2500×10⁶ SMCs)) can be obtained (e.g., isolated) from internal anal sphincter of a subject. For example, a bioengineered IAS construct can comprise about 1.25×10⁶ SMCs. Accordingly, for generation of 8 IAS constructs, about 10×10⁶ SMCs can be isolated, e.g., from an anorectum tissue cell biopsy of a subject.

Additionally, or in the alternative, for generation of bioengineered IAS construct(s) described herein, about 0.2×10⁶ to about 1000×10⁶ NPCs, such as, about 0.2×10⁶ to about 10×10⁶ NPCs (such as, about 0.25×10⁶ to about 10×10⁶ NPCs, about 0.3×10⁶ to about 10×10⁶ NPCs, about 0.35×10⁶ to about 10×10⁶ NPCs, about 0.4×10⁶ to about 10×10⁶ NPCs, about 0.45×10⁶ to about 10×10⁶ NPCs, about 0.5×10⁶ to about 10×10⁶ NPCs, about 0.55×10⁶ to about 10×10⁶ NPCs, about 0.6×10⁶ to about 10×10⁶ NPCs, about 0.65×10⁶ to about 10×10⁶ NPCs, about 0.7×10⁶ to about 10×10⁶ NPCs, about 0.75×10⁶ to about 10×10⁶ NPCs, about 0.8×10⁶ to about 10×10⁶ NPCs, about 0.85×10⁶ to about 10×10⁶ NPCs, about 0.9×10⁶ to about 10×10⁶ NPCs, about 0.95×10⁶ to about 10×10⁶ NPCs, about 1×10⁶ to about 10×10⁶ NPCs, about 1.5×10⁶ to about 10×10⁶ NPCs, about 2×10⁶ to about 10×10⁶ NPCs, about 2.5×10⁶ to about 10×10⁶ NPCs, about 3×10⁶ to about 10×10⁶ NPCs, about 3.5×10⁶ to about 10×10⁶ NPCs, about 4×10⁶ to about 10×10⁶ NPCs, about 4.5×10⁶ to about 10×10⁶ NPCs, about 5×10⁶ to about 10×10⁶ NPCs, about 5.5×10⁶ to about 10×10⁶ NPCs, about 6×10⁶ to about 10×10⁶ NPCs, about 6.5×10⁶ to about 10×10⁶ NPCs, about 7×10⁶ to about 10×10⁶ NPCs, about 7.5×10⁶ to about 10×10⁶ NPCs, about 8×10⁶ to about 10×10⁶ NPCs, about 8.5×10⁶ to about 10×10⁶ NPCs, about 9×10⁶ to about 10×10⁶ NPCs, about 9.5×10⁶ to about 10×10⁶ NPCs (e.g., about 0.2×10⁶ NPCs, 0.25×10⁶ NPCs, 0.3×10⁶ NPCs, 0.35×10⁶ NPCs, 0.4×10⁶ NPCs, 0.45×10⁶ NPCs, 0.5×10⁶ NPCs, 0.55×10⁶ NPCs, 0.6×10⁶ NPCs, 0.65×10⁶ NPCs, 0.7×10⁶ NPCs, 0.75×10⁶ NPCs, 0.8×10⁶ NPCs, 0.85×10⁶ NPCs, 0.9×10⁶ NPCs, 0.95×10⁶ NPCs, 1×10⁶ NPCs, 1.5×10⁶ NPCs, 2×10⁶ NPCs, 2.5×10⁶ NPCs, 3×10⁶ NPCs, 3.5×10⁶ NPCs, 4×10⁶ NPCs, 4.5×10⁶ NPCs, 5×10⁶ NPCs, 5.5×10⁶ NPCs, 6×10⁶ NPCs, 6.5×10⁶ NPCs, 7×10⁶ NPCs, 7.5×10⁶ NPCs, 8×10⁶ NPCs, 8.5×10⁶ NPCs, 9×10⁶ NPCs, 9.5×10⁶ NPCs, or 10×10⁶ NPCs)), about 10×10⁶ to about 100×10⁶ NPCs (such as, about 15×10⁶ to about 100×10⁶ NPCs, about 20×10⁶ to about 100×10⁶ NPCs, about 25×10⁶ to about 100×10⁶ NPCs, about 30×10⁶ to about 100×10⁶ NPCs, about 35×10⁶ to about 100×10⁶ NPCs, about 40×10⁶ to about 100×10⁶ NPCs, about 45×10⁶ to about 100×10⁶ NPCs, about 50×10⁶ to about 100×10⁶ NPCs, about 55×10⁶ to about 100×10⁶ NPCs, about 60×10⁶ to about 100×10⁶ NPCs, about 65×10⁶ to about 100×10⁶ NPCs, about 70×10⁶ to about 100×10⁶ NPCs, about 75×10⁶ to about 100×10⁶ NPCs, about 80×10⁶ to about 100×10⁶ NPCs, about 85×10⁶ to about 100×10⁶ NPCs, about 90×10⁶ to about 100×10⁶ NPCs, about 95×10⁶ to about 100×10⁶ NPCs (e.g., about 15×10⁶ NPCs, 20×10⁶ NPCs, 25×10⁶ NPCs, 30×10⁶ NPCs, 35×10⁶ NPCs, 40×10⁶ NPCs, 45×10⁶ NPCs, 50×10⁶ NPCs, 55×10⁶ NPCs, 60×10⁶ NPCs, 65×10⁶ NPCs, 70×10⁶ NPCs, 75×10⁶ NPCs, 80×10⁶ NPCs, 85×10⁶ NPCs, 90×10⁶ NPCs, 95×10⁶ NPCs, or 100×10⁶ NPCs)), or about 100×10⁶ to about 1000×10⁶ NPCs (such as, about 150×10⁶ to about 1000×10⁶ NPCs, about 200×10⁶ to about 1000×10⁶ NPCs, about 250×10⁶ to about 1000×10⁶ NPCs, about 300×10⁶ to about 1000×10⁶ NPCs, about 350×10⁶ to about 1000×10⁶ NPCs, about 400×10⁶ to about 1000×10⁶ NPCs, about 450×10⁶ to about 1000×10⁶ NPCs, about 500×10⁶ to about 1000×10⁶ NPCs, about 550×10⁶ to about 1000×10⁶ NPCs, about 600×10⁶ to about 1000×10⁶ NPCs, about 6500×10⁶ to about 1000×10⁶ NPCs, about 700×10⁶ to about 1000×10⁶ NPCs, about 750×10⁶ to about 1000×10⁶ NPCs, about 800×10⁶ to about 1000×10⁶ NPCs, about 850×10⁶ to about 1000×10⁶ NPCs, about 900×10⁶ to about 1000×10⁶ NPCs, about 950×10⁶ to about 1000×10⁶ NPCs (e.g., about 150×10⁶ NPCs, 200×10⁶ NPCs, 250×10⁶ NPCs, 300×10⁶ NPCs, 350×10⁶ NPCs, 400×10⁶ NPCs, 450×10⁶ NPCs, 500×10⁶ NPCs, 550×10⁶ NPCs, 600×10⁶ NPCs, 650×10⁶ NPCs, 700×10⁶ NPCs, 750×10⁶ NPCs, 800×10⁶ NPCs, 850×10⁶ NPCs, 900×10⁶ NPCs, 950×10⁶ NPCs, or 1000×10⁶ NPCs)) can be obtained (e.g., isolated) from intestine (e.g., small intestine, such as jejunum) of a subject. For example, a bioengineered IAS construct as described herein can comprise about 0.5×10⁶ NPCs. Accordingly, for generation of 8 IAS constructs, about 4×10⁶ NPCs can be isolated, e.g., from an intestinal tissue cell biopsy of a subject.

### Tissue Biopsy

Sources of SMCs can include anorectum tissue (e.g., internal anal sphincter) of a subject. Accordingly, methods of generating IAS constructs can include steps of obtaining an anorectum tissue cell biopsy from a subject. For example, the method can include obtaining a biopsy from the subject's internal anal sphincter. The anorectum tissue biopsy can be obtained by a laparoscopic procedure and/or operation. For use in the present methods, an anorectum tissue biopsy sample can be about 25 mg to about 500 mg; such as, about 50 mg to about 200 mg, about 75 mg to about 300 mg, about 100 mg to about 400 mg, or about 150 mg to about 500 mg (e.g., about 25 mg; about 50 mg; about 75 mg; about 100 mg; about 125 mg; about 150 mg; about 175 mg; about 200 mg; about 225 mg; about 250 mg; about 275 mg; about 300 mg; about 325 mg; about 350 mg; about 375 mg; about 400 mg; about 425 mg; about 450 mg; about 475 mg; about 500 mg; or more). For example, an anorectum tissue cell biopsy can weigh about 200 mg.

Sources of autologous NPCs can include intestine tissue (e.g., small intestine tissue) of a subject. Accordingly, methods of generating IAS constructs can include steps of obtaining an intestine tissue cell biopsy from a subject. For example, the method can include obtaining a biopsy from the subject's intestinal tissue (e.g., small intestinal tissue). The intestinal tissue biopsy can be obtained by a laparoscopic procedure and/or operation. For example, a small intestine biopsy can include a longitudinal intestinal biopsy on an antimesenteric side of the jejunum. For use in the present methods, an intestinal tissue biopsy sample can be about 25 mg to about 500 mg (e.g., about 25 mg; about 50 mg; about 75 mg; about 100 mg; about 125 mg; about 150 mg; about 175 mg; about 200 mg; about 225 mg; about 250 mg; about 275 mg; about 300 mg; about 325 mg; about 350 mg; about 375 mg; about 400 mg; about 425 mg; about 450 mg; about 475 mg; about 500 mg; or more). For example, an intestinal tissue cell biopsy can weigh about 200 mg.

The biopsies described herein (e.g., the anorectum tissue cell biopsy and the intestinal tissue cell biopsy) can be collected from the same subject. Additionally, or in the alternative, the biopsies described herein can be collected from different subjects.

### Cell Isolation

Methods of generating IAS constructs, as described here, can include steps of isolating SMCs and NPCs from anorectum tissue (e.g., anorectum tissue cell biopsy) and intestinal tissue (e.g., intestinal tissue cell biopsy), respectively. Thus, described herein are methods for processing tissues (e.g., anorectum tissue and/or intestinal tissue), such as biopsied tissues, and culture and expansion of SMCs and NPCs. The anorectum tissues and intestinal tissues can undergo separate processing. Additionally, or in the alternative, the anorectum tissues and intestinal tissues can be processed together. While not wishing to be bound to a theory, it is believed that because the bioengineered IAS constructs can be fabricated from a subject's cells, there is a reduced likelihood of immunologic response and/or rejection in a subject following implantation of the IAS constructs. For example, there may be minimal to no immunologic response or rejection in a subject due to implantation of the IAS constructs provided herein.

### Processing Anorectum Tissue

Processing an anorectum tissue (e.g., an internal anal sphincter tissue) for use in the present methods can include the steps of washing the tissue in an antibiotic solution. The washing can include a washing cycle, which can include shaking at about 5 rpm; about 10 rpm; about 50 rpm; about 100 rpm; about 250 rpm; about 500 rpm or about 1000 rpm or more. The washing cycle can extend for about 5 min; about 10 min; about 15 min; about 20 min; about 25 min; about 30 min; about 35 min; about 40 min; about 45 min; about 50 min; about 55 min; about 60 min; about 2 hours; or about 3 hours; or more. The washing can include a plurality of washing cycles, for example, two cycles, three cycles, four cycles, or more. The antibiotic solution can include one or more of Gentamicin, Vancomycin, Ceftazidime, Amphotericin B, Amikacin, and 2× antibiotic/antimycotic at a concentration of about 5 μg/ml to about 500 μg/ml (e.g., about 10 μg/ml to about 400 μg/ml, about 50 μg/ml to about 300 μg/ml, or about 100 μg/ml to about 200 μg/ml).

Processing of biopsied anorectum tissues can include mincing the washed anorectum tissues into fine pieces. Processing can also include centrifuging the pieces in a disinfecting solution. Processing can further include steps of enzymatically digesting the pieces in an enzyme solution. The enzyme solution can include, for example, 1 mg/ml collagenase DE. The enzymatic digestion can include shaking and/or mixing. For example, the shaking and/or mixing can occur for about 30 minutes; about 45 min; about 60 min; about 2 hours; about 3 hours; about 4 hours; about 5 hours; about 6 hours; about 7 hours; about 8 hours; about 9 hours; about 10 hours; or more. The shaking and/or mixing can occur at about 5 rpm; about 10 rpm; about 50 rpm; about 100 rpm; about 250 rpm; about 500 rpm or about 1000 rpm or more.

The processing can further include centrifuging the digested anorectum tissue. For example, processing can further include steps of enzymatically digesting the centrifuged supernatant in a second enzymatic digest, which can include 1 mg/ml collagenase DE. The enzymatic digestion can include shaking and/or mixing. For example, the shaking and/or mixing can occur for about 30 minutes; about 45 min; about 60 min; about 2 hours; about 3 hours; about 4 hours; about 5 hours; about 6 hours; about 7 hours; about 8 hours; about 9 hours; about 10 hours; or more. The shaking and/or mixing can occur at about 5 rpm; about 10 rpm; about 50 rpm; about 100 rpm; about 250 rpm; about 500 rpm or about 1000 rpm or more. The processing can further include centrifuging the digested anorectum tissue. Processing can further include a plurality of steps of centrifuging the digested anorectum tissue. For example, processing can further include centrifuging the digested anorectum tissue and suspending the pelleted tissue in a SMC growth media. The SMC growth media can include DMEM high glucose supplemented with FBS (e.g., about 1% to about 20% FBS; such as, about 5% to about 20% FBS, about 10% to about 20% FBS, or about 15% to about 20% FBS), L-glutamine (e.g., about 0.5 mM to about 10 mM L-glutamine; such as, about 1 mM to about 10 mM L-glutamine, about 2.5 mM to about 10 mM L-glutamine, about 5 mM to about 10 mM L-glutamine, or about 7.5 mM to about 10 mM L-glutamine), and 1× antibiotic/antimycotic.

SMCs isolated from anorectum tissue biopsy, as described hereinabove, can acquire a spindle-like morphology. Following isolation from anorectum tissue biopsy, the SMCs can be identified and quantified by immunofluorescence (e.g., flow cytometry). For example, flow cytometry can be performed on isolated, cultured SMCs after each isolation. SMCs can be identified by using antibodies directed to SMC marker(s), such as α-smooth muscle actin. For example, SMCs can be incubated with anti-α-smooth muscle actin primary antibodies followed by the appropriate fluorescent secondary antibodies. Cells incubated with fluorescent secondary antibodies can be used as controls. Flow cytometry analysis can demonstrate a high percentage of SMCs expressing muscle-specific antigens, such as, α-smooth muscle actin, indicating the purity of the culture. For example, about 50%-100% (e.g., about 60%-90%, about 70%-80%, about 80%-90%, or about 90%-100% (e.g., about 50%, about 60%, about 70%, about 80%, about 90%, about 95%, or about 99%) of cells isolated from anorectum tissue biopsy can be positive for α-smooth muscle actin.

### Processing Intestinal Tissue

Processing an intestinal tissue (e.g., a small intestinal tissue) biopsy, for use in the present methods, can include steps of washing the biopsy with an antibiotic solution. The washing can include a washing cycle, which can include shaking at about 5 rpm; about 10 rpm; about 50 rpm; about 100 rpm; about 250 rpm; about 500 rpm or about 1000 rpm or more. The washing cycle can extend for about 5 min; about 10 min; about 15 min; about 20 min; about 25 min; about 30 min; about 35 min; about 40 min; about 45 min; about 50 min; about 55 min; about 60 min; about 2 hours; or about 3 hours; or more. Washing can include a plurality of washing cycles, for example, two cycles, three cycles, four cycles, or more. The antibiotic solution can include, for example, one or more of Gentamicin, Vancomycin, Ceftazidime, Amphotericin B, Amikacin, and 2× antibiotic/antimycotic at a concentration of about 5 μg/ml to about 500 μg/ml (e.g., about 10 μg/ml to about 400 μg/ml, about 50 μg/ml to about 300 μg/ml, or about 100 μg/ml to about 200 μg/ml).

Processing of biopsied intestinal tissues can include mincing of the washed intestinal tissues into fine pieces. The processing can include centrifuging the pieces in a disinfecting solution. Processing can further include steps of enzymatically digesting the intestinal tissue pieces in an enzyme solution. The enzyme solution can include a collagenase and another protease, for example, Collagenase HA and BP protease in a concentration of about 5 μg/ml to about 50 μg/ml (e.g., about 10 μg/ml to about 40 μg/ml, about 15 μg/ml to about 30 μg/ml, or about 20 μg/ml to about 25 μg/ml). Additionally, or in the alternative, the enzyme solution can include, for example, a collagenase, e.g., a type II collagenase and a neutral protease, e.g., Dispase II. The enzymatic digestion can include shaking and/or mixing. The shaking and/or mixing can occur for about 30 minutes; about 45 min; about 60 min; about 2 hours; about 3 hours; about 4 hours; about 5 hours; about 6 hours; about 7 hours; about 8 hours; about 9 hours; about 10 hours; or more. The shaking and/or mixing can occur at about 5 rpm; about 10 rpm; about 50 rpm; about 100 rpm; about 250 rpm; about 500 rpm or about 1000 rpm or more.

Processing can further include centrifuging the digested intestinal tissue. For example, processing can further include steps of enzymatically digesting the centrifuged supernatant in a second enzymatic digest, which can include collagenase HA and BP protease in a concentration of about 5 μg/ml to about 50 μg/ml (e.g., about 10 μg/ml to about 40 μg/ml, about 15 μg/ml to about 30 μg/ml, or about 20 μg/ml to about 25 μg/ml). The enzymatic digestion can include shaking and/or mixing. The shaking and/or mixing can occur for about 30 minutes; about 45 min; about 60 min; about 2 hours; about 3 hours; about 4 hours; about 5 hours; about 6 hours; about 7 hours; about 8 hours; about 9 hours; about 10 hours; or more. The shaking and/or mixing can occur at about 5 rpm; about 10 rpm; about 50 rpm; about 100 rpm; about 250 rpm; about 500 rpm or about 1000 rpm or more. Processing can further include centrifuging the digested intestinal tissue and suspending the pelleted tissue in a neural growth media. The neural growth media can include, for example, Phenol Red Free Neural Basal A Medium (ThermoFisher) or a Neurobasal media (Invitrogen) supplemented with one or more of N2 supplement (e.g., about 0.25% to about 5% N2 supplement, such as, about 0.5% to about 5% N2 supplement, about 1% to about 5% N2 supplement, or about 2.5% to about 5% N2 supplement), bFGF (e.g., about 10 ng/ml to about 50 ng/ml bFGF; such as, about 20 ng/ml to about 50 ng/ml bFGF, about 30 ng/ml to about 50 ng/ml bFGF, or about 40 ng/ml to about 50 ng/ml bFGF), EGF (e.g., about 10 ng/ml to about 50 ng/ml EGF; such as, about 20 ng/ml to about 50 ng/ml EGF, about 30 ng/ml to about 50 ng/ml EGF, or about 40 ng/ml to about 50 ng/ml EGF), L-Glutamine (e.g., about 0.5 mM to about 10 mM L-glutamine; such as, about 1 mM to about 10 mM L-glutamine, about 2.5 mM to about 10 mM L-glutamine, about 5 mM to about 10 mM L-glutamine, or about 7.5 mM to about 10 mM L-glutamine), and 1× antibiotic/antimycotic.

NPCs isolated from intestinal tissue biopsy, as described hereinabove, can be characterized (e.g., identified and/or quantified) by immunofluorescence (e.g., immunocytochemistry). In particular, immunocytochemistry can be performed on isolated, cultured NPCs after each isolation. NPCs can be identified by using antibodies directed to NPC marker(s), such as p75^{NTR}. For example, NPCs can be stained with p75^{NTR}, and smoothelin and/or Oct4 can be used as negative control(s). Immunocytochemical analysis can demonstrate a high percentage of NPCs expressing p75^{NTR}, indicating the purity of the culture. For example, about 50%-100% (e.g., about 60%-90%, about 70%-80%, about 80%-90%, or about 90%-100% (e.g., about 50%, about 60%, about 70%, about 80%, about 90%, about 95%, about 99%, or about 100%) of cells isolated from intestinal tissue biopsy can be positive for p75^{NTR}. Additionally, or in the alternative, about 50%-100% (e.g., about 60%-90%, about 70%-80%, about 80%-90%, or about 90%-100% (e.g., about 50%, about 60%, about 70%, about 80%, about 90%, about 95%, about 99%, or about 100%) of cells isolated from intestinal tissue biopsy can be negative for smoothelin and/or Oct4.

### Cell Expansion and Harvest

Methods of generating IAS constructs, as described here, can include steps of expanding and harvesting SMCs and NPCs.

Methods for expansion and harvest of SMCs can include steps of monitoring SMC culture counts. SMC count can be monitored and the SMCs can be harvested when the cells reach about 40% to about 100% confluence (e.g., about 40% confluence; about 50% confluence; about 60% confluence; about 70% confluence; about 80% confluence; about 90% confluence; or about 100% confluence). For example, the SMCs can be harvested when the cells reach approximately 90% confluence. The SMCs can be harvested using cell-dissociation enzymes, such as TrypeFE reagents. Harvesting can release the SMCs from the plate. The SMCs can then be sub-cultured to expand to a desired number of cells. Thus, the expansion and harvest of SMCs can further include a step of cell counting. The aforementioned steps for expansion and harvest of SMCs can be repeated until cell counts reach at least 250,000; at least 500,000; at least 1 million; at least 2 million; at least 2.5 million; at least 3 million; at least 3.5 million; at least 4 million; at least 4.5 million; at least 5 million; at least 7.5 million; at least 10 million; at least 15 million; at least 20 million; at least 25 million; at least 30 million; at least 35 million; at least 40 million; at least 45 million; at least 50 million; or more. For use in the present methods, SMCs can be cultured (e.g., cultured, expanded, and harvested) for one or more weeks (e.g., about 2 weeks, 3 weeks, 4 weeks, 5 weeks, 6 weeks, 7 weeks, 8 weeks, 9 weeks, 10 weeks, 11 weeks, 12 weeks, or more).

Methods for the expansion and harvest of NPCs can include steps of monitoring NPC counts. NPCs can be monitored under a microscope for the formation of cell clusters. The NPCs can be centrifuged when NPC cluster density reaches about 40% to about 100% confluence (e.g., about 40% confluence; about 50% confluence; about 60% confluence; about 70% confluence; about 80% confluence; about 90% confluence; or about 100% confluence). For example, NPCs can be centrifuged when the cluster density reaches about 70% confluence. Methods for expansion and harvest of NPCs can further include digesting the cell pellet, for example in Accutase, followed by steps of neutralizing in a media (e.g., Phenol Red Free Neural Basal A medium, Neuro Basal medium (Invitrogen) or Dulbecco's Modified Eagle Medium-F12 medium). The method can further include a step of counting NPCs. The aforementioned steps for expansion and harvest of NPCs can be repeated until cell counts reach at least 250,000; at least 500,000; at least 1 million; at least 2 million; at least 2.5 million; at least 3 million; at least 3.5 million; at least 4 million; at least 4.5 million; at least 5 million; at least 7.5 million; at least 10 million; at least 15 million; at least 20 million; at least 25 million; at least 30 million; at least 35 million; at least 40 million; at least 45 million; at least 50 million; or more. For use in the present methods, NPCs can be cultured (e.g., cultured, expanded, and harvested) for one or more weeks (e.g., about 2 weeks, 3 weeks, 4 weeks, 5 weeks, 6 weeks, 7 weeks, 8 weeks, 9 weeks, 10 weeks, 11 weeks, 12 weeks, or more).

### Single-Layer Gel Matrix

Methods for generating IAS constructs can further include steps of preparing a single-layer gel matrix. For the preparation of a single-layer gel matrix, a cell mix can be prepared that comprises the SMCs and the NPCs in a collagen/laminin gel. For example, neural progenitor cells isolated from a patient are collected, suspended in collagen/laminin gel, and mixed with smooth muscle cells (e.g., from the same patient). For example, the NPCs (e.g., NPCs isolated by the aforementioned methods) can be suspended in collagen/laminin gel, and mixed with the SMCs (e.g., SMCs isolated by the aforementioned methods) to obtain a cell mix. The cell mix can then be seeded on a mold (e.g., around a central post of a mold) as a single-layer gel matrix.

The single-layer gel matrix can comprise laminin and collagen in a ratio of about 1:1 to about 1:1000; such as, about 1:1 to about 1:10 (such as, in a ratio of about 1:2 to about 1:10, about 1:3 to about 1:10, about 1:4 to about 1:10, about 1:5 to about 1:10, about 1:6 to about 1:10, about 1:7 to about 1:10, about 1:8 to about 1:10, or about 1:9 to about 1:10 (e.g., in a ratio of about 1:1, 1:2, 1:3, 1:4, 1:5, 1:6, 1:7, 1:8, 1:9, or 1:10)), about 1:10 to about 1:100 (such as, in a ratio of about 1:20 to about 1:100, about 1:30 to about 1:100, about 1:40 to about 1:100, about 1:50 to about 1:100, about 1:60 to about 1:100, about 1:70 to about 1:100, about 1:80 to about 1:100, or about 1:90 to about 1:100 (e.g., in a ratio of about 1:10, 1:20, 1:30, 1:40, 1:50, 1:60, 1:70, 1:80, 1:90, or 1:100)), about 1:100 to about 1:1000 (such as, in a ratio of about 1:200 to about 1:1000, about 1:300 to about 1:1000, about 1:400 to about 1:1000, about 1:500 to about 1:1000, about 1:600 to about 1:1000, about 1:700 to about 1:1000, about 1:800 to about 1:1000, or about 1:900 to about 1:1000 (e.g., in a ratio of about 1:100, 1:200, 1:300, 1:400, 1:500, 1:600, 1:700, 1:800, 1:900, or 1:1000)). For example, the single-layer gel matrix can comprise laminin and collagen in a ratio of about 1:5 to about 1:10, about 1:5 to about 1:50, about 1:5 to about 1:100, about 1:5 to about 1:500, or about 1:5 to about 1:1000.

The cell mix and/or the single-layer gel matrix can comprise NPCs and SMCs in a ratio of about 1:1 to about 1:1000; such as, in a ratio of about 1:1 to about 1:10 (such as, in a ratio of about 1:2 to about 1:5, about 1:3 to about 1:6, about 1:4 to about 1:7, about 1:5 to about 1:8, about 1:6 to about 1:9, or about 1:7 to about 1:10 (e.g., in a ratio of about 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10)), about 1:10 to about 1:100 (such as, in a ratio of about 1:20 to about 1:50, about 1:30 to about 1:60, about 1:40 to about 1:70, about 1:50 to about 1:80, about 1:60 to about 1:90, or about 1:70 to about 1:100 (e.g., in a ratio of about 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or 1:100)), or about 1:100 to about 1:1000 (such as, in a ratio of about 1:200 to about 1:500, about 1:300 to about 1:600, about 1:400 to about 1:700, about 1:500 to about 1:800, about 1:600 to about 1:900, or about 1:700 to about 1:1000 (e.g., in a ratio of about 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1000)).

As used herein, “collagen” can refer to collagen I or collagen II or collagen III or collagen IV. Collagen I can include collagen I or collagen I compositions derived from cell culture, animal tissue, or recombinant means, and can be derived from human, murine, porcine, or bovine sources. Additionally, collagen I can comprise collagen I or collagen I compositions that do not include a collagen I fragment, e.g., including essentially only a complete collagen I protein. Collagen II can comprise collagen II or collagen II compositions derived from cell culture, animal tissue, or recombinant means, and can be derived from human, murine, porcine, or bovine sources. Additionally, collagen II can comprise collagen II or collagen II compositions that do not include a collagen II fragment, e.g., including essentially only a complete collagen II protein.

Collagen III can comprise collagen III or collagen III compositions derived from cell culture, animal tissue, or recombinant means, and can be derived from human, murine, porcine, or bovine sources. Additionally, collagen III can comprise collagen III or collagen III compositions that do not include a collagen III fragment, e.g., including essentially only a complete collagen III protein.

Collagen IV can comprise collagen IV or collagen IV compositions derived from cell culture, animal tissue, or recombinant means, and can be derived from human, murine, porcine, or bovine sources. Additionally, collagen IV can comprise collagen IV or collagen IV compositions that do not include a collagen IV fragment, e.g., including essentially only a complete collagen IV protein.

As used herein, “laminin” can refer to laminin, laminin fragments, laminin derivatives, laminin analogs, or laminin compositions derived from cell culture, recombinant means, or animal tissue. Laminin can be derived from human, murine, porcine, or bovine sources. “Laminin” can refer to laminin or laminin compositions comprising laminin-1, laminin-2, laminin-4, or combinations thereof. Additionally, “laminin” can refer to laminin or laminin compositions that do not include a laminin fragment, e.g., including essentially only a complete laminin protein.

Accordingly, the methods described herein can include steps of suspending an expanded culture of NPCs (e.g., NPCs expanded and harvested by the aforementioned methods) in a gel mixture. The gel mixture can include medical-grade collagen or clinical-grade collagen or research-grade collagen recombinant laminin, Phenol Red Free Neural Basal A medium, or Dulbecco's Modified Eagle Medium, and water. The NPC suspension can then be mixed with SMCs or SMCs suspension can be mixed with NPCs (e.g., SMC pellet obtained by expansion and harvest of SMCs) to obtain a cell mix. The cell mix can then be seeded as a single-layer gel matrix onto a prepared mold (e.g., around a central post of a mold). Seeding or laying of a single-layer gel matrix onto a prepared mold can include laying the cell mix in a substantially circular shape on the mold. For example, seeding or laying of a single-layer gel matrix onto a prepared mold can include laying the cell mix around a central post of the mold. The method can include steps of gently swirling the mold to ensure complete coverage of the mold. The cell mix can then be allowed to gel on the mold (e.g., around a central post of a mold). The cell mix can be allowed to gel at about 20-40° C. (e.g., about 20-25° C., 25-30° C., 30-35° C., or 35-40° C.; such as, about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.) and about 1-10% CO₂ (e.g., about 1% CO₂, 2% CO₂, 3% CO₂, 4% CO₂, 5% CO₂, 6% CO₂, 7% CO₂, 8% CO₂, 9% CO₂, or 10% CO₂) for about 10-20 minutes, about 20-30 minutes, about 30-60 minutes, about 60-90 minutes, about 90-120 minutes, about 120-180 minutes, or more. Additionally, or in the alternative, the cell mix can be allowed to gel for at least 10 minutes (e.g., at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120, at least 180 minutes, or more) at about 20-40° C. (e.g., about 20-25° C., 25-30° C., 30-35° C., or 35-40° C.; such as, about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.) and about 1-10% CO₂ (e.g., about 1% CO₂, 2% CO₂, 3% CO₂, 4% CO₂, 5% CO₂, 6% CO₂, 7% CO₂, 8% CO₂, 9% CO₂, or 10% CO₂).

Following gelation, differentiation media can be added to the single-layer gel matrix on the mold. The differentiation media can include, for example, Neurobasal-A media (Invitrogen) supplemented with B27 supplement, FBS (e.g., about 0.5% to about 20% FBS; such as, about 1% to about 20% FBS, about 2% to about 20% FBS, about 5% to about 20% FBS, about 10% to about 20% FBS, or about 15% to about 20% FBS), and Gentamicin or any other antibiotics. The single-layer gel matrix can then be incubated in the differentiation media at about 20-40° C. (e.g., about 20-25° C., 25-30° C., 30-35° C., or 35-40° C.; such as, about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.) and about 1-10% CO₂ (e.g., about 1% CO₂, 2% CO₂, 3% CO₂, 4% CO₂, 5% CO₂, 6% CO₂, 7% CO₂, 8% CO₂, 9% CO₂, or 10% CO₂) to allow maturation of the gel matrix. Maturation of of the gel matrix can include remodeling of the gel matrix (e.g., into tissue). During remodeling, the liquid gel becomes tissue with cells lined up around the central post of the mold.

Maturation (e.g., remodeling) of the single-layer gel matrix can induce differentiation of the NPCs, and the SMCs can contract into a ring-like structure around the post, thus forming an innervated IAS construct having directionally oriented SMCs. An IAS construct can be formed (e.g., a single-layer gel matrix can mature into an IAS construct) within 1-15 days, such as within 1-3 days, 3-5 days, 5-7 days, 7-9 days, 9-11 days, 11-13 days, 13-15 days, 2-15 days, 3-15 days, 4-15 days, 5-15 days, 6-15 days, 7-15 days, 8-15 days, 9-15 days, 10-15 days, 11-15 days, 12-15 days, 14-15 days, 2-12 days, 3-12 days, 4-12 days, 5-12 days, 6-12 days, 7-12 days, 8-12 days, 9-12 days, 10-12 days, or 11-12 days (e.g., within 1 day, within 2 days, within 3 days, within 4 days, within 5 days, within 6 days, within 7 days, within 8 days, within 9 days, within 10 days, within 11 days, within 12 days, within 13 days, within 14 days, or within 15 days). For example, an IAS construct can be formed in 7 days, 8 days, 9 days, 10 days, 11 days, or 12 days. The differentiation medium can be changed every 1 day, every 2 days, every 3 days, every 4 days, every 5 days, or every 6 days during maturation of the single-layer gel matrix. For example, a single-layer gel matrix can be cultured for 12 days in the differentiation medium with a change of medium every fourth day.

Silicone Plate and/or Mold

As used herein, the term “mold” is intended to encompass any culture plate or substrate suitable for receiving, without limitation, a cell mix comprising SMCs and NPCs in a single layer (e.g., a single-layer gel matrix), and guiding their integration into a circular construct. In certain embodiment, the mold can be a plate with sides and a central post. An exemplary mold for use in the methods of the present disclosure is described in U.S. Pat. No. 7,368,279 B2.

Methods for generating IAS constructs can further include steps of providing molds. The step of providing a mold can further include preparing a mold. The molds for use in the present methods can be formed from or fabricated from silicone, or any other inert material (e.g., polymer, ceramic, or metal). For example, the molds can be formed from or fabricated from medical grade silicone. The step of forming a mold can include mixing an elastomer with a curing reagent, pouring the mixture on plates (e.g., culture plates), and curing the plates. Additionally, the step of forming a mold can include lining a plate with a layer of silicone; preparing a silicone post; punching a post hole in the plate; and curing the silicone post in the post hole to fix the post to the plate, thereby forming the mold. The step of curing can be heating. Additionally, or in the alternative, the step of curing can be UV exposure. Once formed, the molds can be sterilized.

The size of a mold can depend on the size of the construct to be fabricated. For example, a mold can be characterized by its diameter and/or width. The diameter of a mold can be about 30 mm to about 100 mm (e.g., about 40 mm to about 100 mm, about 50 mm to about 100 mm, about 60 mm to about 100 mm, about 70 mm to about 100 mm, about 80 mm to about 100 mm, or about 90 mm to about 100 mm). For example, the diameter of a mold can be about 30 mm; about 35 mm; about 40 mm; about 45 mm; about 50 mm; about 55 mm; about 60 mm; about 65 mm; about 70 mm; about 75 mm; about 80 mm; about 85 mm; about 90 mm; about 95 mm; or about 100 mm. A mold can include a central post. For example, a mold can be characterized by the diameter of the central post. The diameter of a central post of a mold can be about 5 mm to about 40 mm (e.g., about 10 mm to about 40 mm, about 15 mm to about 40 mm, about 20 mm to about 40 mm, about 25 mm to about 40 mm, about 30 mm to about 40 mm, about 35 mm to about 40 mm, about 5 mm to about 25 mm, about 10 mm to about 25 mm, about 15 mm to about 25 mm, or about 20 mm to about 25 mm). For example, the diameter of a central post of the mold can be about 5 mm; about 8 mm; about 10 mm; about 12 mm; about 14 mm; about 15 mm; about 16 mm; about 18 mm; about 20 mm; about 22 mm; about 24 mm; about 25 mm; about 26 mm; about 28 mm; about 30 mm; about 32 mm; about 34 mm; about 35 mm; about 36 mm; about 38 mm; or about 40 mm.

### IAS Construct

An innervated IAS construct can be generated by the methods described herein. For example, an IAS construct can comprise a functional neural network of NPCs with concentrically aligned SMCs. An IAS construct (e.g., an innervated IAS construct) of the present disclosure can comprise a network of autologous collagen, such as, a network of autologous human collagen. Following maturation of a single layer gel matrix by the aforementioned methods, about 50% (e.g., about 55%, about 60%, about 65%, about 70%, about 75%, about 80%, about 85%, about 90%, about 95%, about 96%, about 97%, about 98%, about 99%, or more) of the initial bovine collagen content can be replaced by human collagen. Accordingly, an IAS construct can comprise less than 10% (e.g., less than 9.5%, less than 9%, less than 8.5%, less than 8%, less than 7.5%, less than 7%, less than 6.5%, less than 6%, less than 5.5%, less than 5%, less than 4.5%, less than 4%, less than 3.5%, less than 3%, less than 2.5%, less than 2%, less than 1.5%, less than 1%, or less than 0.5%) bovine collagen.

An IAS construct (e.g., an innervated IAS construct) of the present disclosure can comprise a ring structure with a central open lumen. The central open lumen of an IAS construct can have a diameter of about 5 mm to about 50 mm, such as, about 10 mm to about 50 mm, about 15 mm to about 45 mm, about 20 mm to about 40 mm, about 25 mm to about 35 mm, about 20 mm to about 35 mm, about 20 mm to about 30 mm, or about 20 mm to about 25 mm (e.g., about 5 mm, 10 mm, 15 mm, 20 mm, 25 mm, 30 mm, 35 mm, 40 mm, 45 mm, or 50 mm). For example, an IAS construct can comprise a ring structure with a 20-mm diameter central open lumen.

An IAS construct (e.g., an innervated IAS construct) of the present disclosure can have a surface area of about 100 mm² to about 200 mm², such as about 110 mm² to about 190 mm², about 120 mm² to about 180 mm², about 130 mm² to about 170 mm², about 140 mm² to about 160 mm², or about 140 mm² to about 150 mm² (e.g., about 100 mm², about 110 mm², about 120 mm², about 130 mm², about 140 mm², about 150 mm², about 160 mm², about 170 mm², about 180 mm², about 190 mm², or about 200 mm²). For example, an IAS construct can have a surface area of about 140.90 mm² (e.g., a surface area of about 140.90±0.85 mm²).

An IAS construct (e.g., an innervated IAS construct) of the present disclosure can have a volume of about 100 mm³ to about 200 mm³, such as, about 110 mm³ to about 190 mm³, about 120 mm³ to about 180 mm³, about 130 mm³ to about 170 mm³, about 140 mm³ to about 160 mm³, or about 150 mm³ to about 160 mm³ (e.g., about 100 mm³, about 110 mm³, about 120 mm³, about 130 mm³, about 140 mm³, about 150 mm³, about 160 mm³, about 170 mm³, about 180 mm³, about 190 mm³, or about 200 mm³). For example, an IAS construct can have a volume of about 153.87 mm³ (e.g., a volume of about 153.87±0.62 mm³).

An IAS construct (e.g., an innervated IAS construct) of the present disclosure can have a thickness of about 1.0 mm to about 2.0 mm, such as, about 1.1 mm to about 1.9 mm, about 1.2 mm to about 1.8 mm, about 1.3 mm to about 1.7 mm, about 1.4 mm to about 1.6 mm, about 1.1 mm to about 2.0 mm, about 1.2 mm to about 2.0 mm, about 1.3 mm to about 2.0 mm, about 1.4 mm to about 2.0 mm, about 1.5 mm to about 2.0 mm, about 1.6 mm to about 2.0 mm, about 1.7 mm to about 2.0 mm, about 1.8 mm to about 2.0 mm, or about 1.9 mm to about 2.0 mm (e.g., about 1.0 mm, 1.1 mm, 1.2 mm, 1.3 mm, 1.4 mm, 1.5 mm, 1.6 mm, 1.7 mm, 1.8 mm, 1.9 mm, or 2.0 mm). For example, an IAS construct can have a thickness of about 1.92 mm (e.g., a thickness of about 1.92±0.01 mm).

An IAS construct (e.g., an innervated IAS construct) of the present disclosure can have a height of about 1.0 mm to about 3.0 mm, such as, about 1.1 mm to about 2.9 mm, about 1.2 mm to about 2.8 mm, about 1.3 mm to about 2.7 mm, about 1.4 mm to about 2.6 mm, about 1.5 mm to about 2.5 mm, or about 2.0 mm to about 2.5 mm (e.g., about 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, or 3.0 mm). For example, an IAS construct can have a height of about 2.44 mm (e.g., a height of about 2.44±0.01 mm).

Muscle function (e.g., functions of SMCs) and neural function (e.g., functions of NPCs) of an IAS construct (e.g., an innervated IAS construct) of the present disclosure can be tested by methods described in the Examples section. Briefly, functions of SMCs in an IAS construct can be tested by assessing smooth muscle contraction in response to KCl-induced depolarization. Functions of NPCs in an IAS construct can be tested by using electrical field stimulation (EFS) and assessing relaxation response. IAS constructs of the present disclosure can maintain muscle function and neural function at a level that is comparable to control conditions (e.g., compared to muscle function and neural function of IAS constructs at a temperature of about 37° C.) when the IAS constructs are stored for up to 12 hours (e.g., up to 18 hours, up to 24 hours, up to 36 hours, up to 48 hours, up to 60 hours, up to 72 hours, or more). Additionally, or in the alternative, IAS constructs of the present disclosure can maintain muscle function and neural function at a level that is comparable to control conditions (e.g., compared to muscle function and neural function of IAS constructs at a temperature of about 37° C.) when the IAS constructs are stored at a temperature of about 15° C. to about 37° C. (e.g., at a temperature of about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., or 37° C.). For example, IAS constructs can maintain muscle function and neural function at a level that is comparable to control conditions when the IAS constructs are stored for up to 48 hours at a temperature of about 15° C. to about 37° C. The stability of IAS constructs can be determined by assessing the muscle function and neural function of the constructs. Accordingly, IAS constructs of the present disclosure can be stable when stored for up to 48 hours at a temperature of about 15° C. to about 37° C.

### Method of Treatment

The present methods can be used for treating fecal incontinence in a subject by implanting to the subject one or more IAS constructs described hereinabove. Fecal incontinence (FI) is the inability to control bowel movements, causing stool (feces) to leak unexpectedly from the rectum. Fecal incontinence, also known as bowel incontinence, can range from an occasional leakage of stool while passing gas to a complete loss of bowel control. Fecal incontinence can refer to loss of control sufficient to have stool in a subject's undergarment(s) and/or sufficient to require the changing of the undergarment(s). As used herein, the term “subject” refers to any living organism, including, but not limited to, humans; nonhuman primates such as chimpanzees and other apes and monkey species; farm animals such as cattle, sheep, pigs, goats, and horses; domestic mammals such as dogs and cats; laboratory animals including rodents such as mice, rats, rabbits, and guinea pigs, and the like. The term does not denote a particular age or sex. As used herein, the term “treating” refers to partially or completely alleviating, ameliorating, relieving, inhibiting, preventing (for at least a period of time), delaying the onset of, reducing severity of, reducing frequency of and/or reducing incidence of one or more symptoms or features of fecal incontinence, e.g., to reduce or eliminate frequency (e.g., number) or severity (e.g., volume) of fecal leakage. For example, following implantation of the IAS constructs described herein, the number and/or volume of fecal incontinence incidences (e.g., incontinence accidents) in a subject may reduce by about 30% or more (e.g., about 35% or more, about 40% or more, about 45% or more, about 50% or more, about 55% or more, about 60% or more, about 65% or more, about 70% or more, about 75% or more, about 80% or more, about 85% or more, about 90% or more, or about 95% or more). Frequency (e.g., number) or severity (e.g., volume) of fecal incontinence incidences may reduce in a subject within 12 months (e.g., within 11 months, within 10 months, within 9 months, within 8 months, within 7 months, within 6 months, within 5 months, within 4 months, within 3 months, within 2 months, or within 1 month) of implantation of the IAS constructs.

Subjects experiencing 2 or more (e.g., 3 or more, 4 or more, 5 or more, 6 or more, 7 or more, 8 or more, 9 or more, or 10 or more) fecal incontinence incidences per 2-week period for 6 months or more (e.g., 7 months, 8 months, 9 months, 10 months, 11 months, 12 months, 18 months, 24 months, or more) can be implanted with IAS construct(s) of the present disclosure for treating the fecal incontinence. In particular, subjects with severe passive fecal incontinence can be implanted with IAS construct(s) of the present disclosure for treating the fecal incontinence. For example, subjects with fecal incontinence can be implanted with IAS construct(s) for treating the fecal incontinence if they have failed all medical and surgical therapy for fecal incontinence and are being considered for a colostomy. Failed standard medical and surgical therapies for fecal incontinence can include:
(i) Failure of bulking agents: Bulking agents (e.g., Citrucel or Metamucil) may fail to decrease the frequency of fecal incontinence episodes to 4 or fewer episodes per two-week period after 4 weeks of treatment.
(ii) Failure of antidiarrheal agents: Antidiarrheal agents (e.g., Imodium or Lomotil) may decrease fecal incontinence or produce constipation but fail to decrease the frequency of fecal incontinence episodes to 4 or fewer episodes per two-week period after 4 weeks of treatment.
(iii) Failure of biofeedback training: Biofeedback training may fail to reduce the frequency of fecal incontinence episodes to 4 or fewer episodes per two-week period after 4 weeks of treatment. Treatment failure can be a lack of response to the intervention after 3 months of treatment.
(iv) Failure of Sacral nerve stimulation (SNS): SNS may fail to decrease the frequency of fecal incontinence episodes to 4 or fewer episodes per two-week period after 2 months or more of treatment. SNS requires a two week period for two surgeries, trial implantation and then permanent implantation. During the two week period, patients can see a change in fecal incontinence symptoms secondary to SNS implantation that informs the decision to proceed with permanent implantation. After permanent implantation, the settings of the SNS can be manipulated to optimize fecal control and minimize patient side effects. Two months allows time for the two surgeries to occur and SNS setting changes to ensure optimization of FI. Failure of SNS treatment may also include patients who have SNS explantation due to complications or side effects. Finally, SNS failure can include patients who fail test stimulation and do not undergo chronic implantation.
(v) Failure of Sphincteroplasty: Sphincteroplasty failure will have occurred if the FI episode frequency is four or more episodes per two-week period, 12 months or more after surgical repair.
(vi) Failure of Anorectal manometry (ARM) testing within one year: ARM must show low IAS pressure and presence of the Recto Anal Inhibitory Reflex (RAIR). Low IAS pressure is defined as <50 mmHg during the anorectal motility exam performed with a high-resolution catheter (Given, Atlanta, GA). Normal IAS pressure range is 50 to 100 mmHg.

At least one IAS construct (e.g., at least two IAS constructs, at least three IAS constructs, at least four IAS constructs, at least five IAS constructs, at least six IAS constructs, at least seven IAS constructs, at least eight IAS constructs, at least nine IAS constructs, at least ten IAS constructs, or more) can be implanted in a subject for treating fecal incontinence. Additionally, or in the alternative, a plurality of IAS constructs, for example, more than one IAS construct, (e.g., two IAS constructs, three IAS constructs, four IAS constructs, five IAS constructs, six IAS constructs, seven IAS constructs, eight IAS constructs, nine IAS constructs, ten IAS constructs, eleven IAS constructs, twelve IAS constructs, thirteen IAS constructs, fourteen IAS constructs, fifteen IAS constructs, or more) can be implanted in a subject for treating fecal incontinence. For example, fecal incontinence in a subject can be treated by implanting in the subject four IAS constructs of the present disclosure.

The implantation of the one or more IAS construct can be through a circumferential dissection around the anorectum of a subject. For example, one, two, three, four, or more IAS constructs can be implanted through a circumferential dissection around the anorectum of a subject.

Implanting one or more IAS constructs of the present disclosure can be useful for treating fecal incontinence in a subject who has been diagnosed with fecal incontinence and/or is showing one or more symptoms of fecal incontinence. Implantation of one or more IAS constructs can partially or completely alleviate, ameliorate, relieve, inhibit, prevent (for at least a period of time), reduce the severity of, reduce the frequency of and/or reduce the incidence of one or more symptoms or features of fecal incontinence in such subjects. Additionally, or in the alternative, implanting one or more IAS constructs of the present disclosure can be useful for delaying the onset of fecal incontinence in a subject who: (i) does not yet exhibit symptoms, signs, or characteristics of fecal incontinence; and/or (ii) exhibits only early symptoms, signs and/or characteristics of fecal incontinence; and/or (iii) has been identified to be at risk for developing fecal incontinence. Additionally, or in the alternative, implanting one or more IAS constructs of the present disclosure can be useful for decreasing the risk of developing pathology associated with fecal incontinence in a subject who: (i) does not yet exhibit symptoms, signs, or characteristics of fecal incontinence; and/or (ii) exhibits only early symptoms, signs and/or characteristics of fecal incontinence; and/or (iii) has been identified to be at risk for developing fecal incontinence.

One or more IAS constructs of the present disclosure can be implanted in a subject (e.g., as part of an intervention) after the subject develops one or more symptoms, signs, and/or characteristics of fecal incontinence. Additionally, or in the alternative, one or more IAS constructs of the present disclosure can be implanted in a subject (e.g., as part of prophylactic treatment) before the subject develops one or more symptoms, signs, and/or characteristics of fecal incontinence.

### EXAMPLES

The disclosure is further described in the following examples, which do not limit the scope of the methods described in the claims.

### Example 1. Formulation and Development of Internal Anal Sphincter (IAS) Constructs

### Background and Rationale

The internal anal sphincter (IAS) constructs generated in this Example comprise two kinds of autologous cells: (1) Circular smooth muscle cells (SMC) isolated from human internal anal sphincter (IAS), and (2) Enteric neurospheres (neural progenitor cells (NPC)) isolated from the human small intestine (jejunum). Enteric NPCs were co-cultured with IAS circular smooth muscle cells using layered hydrogels and allowed to form circular, intrinsically innervated IAS constructs. These exemplary constructs will be referred to herein as BioSphincters. These BioSphincters were used as additive implants to treat Fecal Incontinence (FI) in the non-clinical large animal studies (Bohl et al., 2017; Dadhich et al., 2019; Zakhem et al., 2019).

The process of isolation and engineering of the Sphincters are summarized in FIG. 1. FIG. 2 is a schematic representation of a process flow chart describing the details of the manufacturing and release of a BioSphincter. FIG. 3 illustrates the engineering process of a BioSphincter using IAS smooth muscle cells and neural progenitor cells.

### Process Description

The process to develop a BioSphincter construct consists of the following five steps:
Collection of autologous smooth muscle cells (SMCs) from a patient's IAS and autologous neural progenitor cells from a patient's small intestine
Expansion of these SMCs and NPCs in culture
Manufacturing BioSphincter by mixing these cells and suspending them in a single-layer hydrogel matrix
In-process and release testing to demonstrate that the final product meets all specifications.
Packaging of the final BioSphincter construct such that they maintain their shape, viability, and functionality; then transporting the BioSphincter construct to the clinical facility

The entire manufacturing process takes about 6-12 weeks (FIG. 1). The cells get ready for bioengineering of BioSphincter in 6 weeks. After the schedule of Implantation Surgery of the patients, the final BioSphincters are bioengineered, which takes 12 days. Therefore, the range of the entire manufacturing process is kept as 6-12 weeks.

Collection of Small Intestine Biopsy and IAS Biopsy from the Hospital

Two biopsies are collected from the same patient. First, a biopsy from the small intestinal tissue (≥200 mg) is obtained laparoscopically and used to provide autologous NPC for the patient's BioSphincter construct. The second, a biopsy from the IAS (≥200 mg) is obtained through surgical retrieval and provides autologous SMC for the patient's BioSphincter construct.

### Tissue Collection and Transport to Manufacturing Facility

The patient's small intestinal tissue biopsy and IAS tissue biopsy are placed into labeled biopsy containers containing cold transport medium (Hank's balanced salt solution (HBSS) with 50 μg/ml Gentamicin) immediately following its collection and maintained at refrigerated temperature (2-8° C.). The container is sealed and placed into a qualified 2-8° C. shipper. The shipper is then transported to the manufacturing facility within 24 hrs.

### Tissue Processing—Dissection and Enzymatic Digestion

Upon arrival at the manufacturing facility, biopsy tissues are processed immediately or held at 2-8° C. not to exceed 48 hours post-harvest. Development studies conducted with analogous biopsy tissue samples stored in a monitored refrigerator at 2-8° C. for 48 hours before processing confirmed the stability of the tissue for up to 48 hours post-receipt. Biopsy tissues are processed separately, but in parallel, on the same day by two process engineers in an ISO 7 room. Transport media are collected from each biopsy container for endotoxin testing.

### Neural Progenitor Cell Isolation

The small intestine biopsy is taken out of the transport solution container and washed with antibiotic solution (HBSS with 240 μg/ml Gentamicin, 160 μg/ml Vancomycin, 500 μg/ml Ceftazidime, 5.4 μg/ml Amphotericin B and 200 μg/ml Amikacin) multiple times with shaking (100 rpm) for at least 30 min each wash. The biopsy tissue is weighed to determine the required volume of digestion solution, then washed multiple times in disinfection solution (HBSS with 5 μg/ml Gentamicin) for 2 min each wash at room temperature.

The tissue is then placed on a sterile dish and cleaned of any fat or blood vessels with sterile scissors or scalpel. Using a sterile blade, the biopsy tissue is minced into fine pieces and then washed multiple times in disinfection solution at room temperature using centrifugation at 600×g for 5 min each wash. Following the last wash, the minced tissue is incubated with digestion solution (18.5 μl/ml collagenase HA and 26.1 μl/ml BP protease) for 1 hour with shaking (100 rpm) at 37° C.

The digested tissue is centrifuged at 400×g for 5 minutes, after which the supernatant is passed through a 70 μm cell strainer into a new sterile conical tube (appropriately labeled) and stored at 2-8° C. The remaining pellet is subjected to a second enzymatic digest (18.5 μl/ml collagenase HA and 26.1 μl/ml BP protease) for approximately 45 minutes with shaking (100 rpm) at 37° C.

Following the second digest, the remaining tissue is centrifuged at 400×g for 5 minutes and the supernatant is passed through a 70 μm cell strainer into a new sterile conical tube (appropriately labeled). Both tubes that contain the 70 μm suspension are combined into one conical tube. The cells in the combined suspension are pelleted and washed multiple times by resuspending them in disinfection solution followed by centrifugation for 10 min at 2000×g; 15° C. A portion of the last wash is collected for sterility testing.

The cell pellet is then resuspended in warm neural growth media by gentle pipetting. The dissociated pellet is passed through a 40 μm cell strainer into a new conical tube to select NPC based on size. The filtered cells are plated into non-tissue culture treated vessels and cultured for approximately 4 weeks in a humidified incubator at 37° C. with 7% CO₂. Growth is monitored approximately every other day by phase contrast microscopy and the cultures are supplemented once a week with neural progenitor cell growth media.

### Smooth Muscle Cell Isolation

The IAS biopsy is taken out of the transport solution tube and washed in antibiotic solution (HBSS with 240 μg/ml Gentamicin, 160 μg/ml Vancomycin, 500 μg/ml Ceftazidime, 5.4 μg/ml Amphotericin B and 200 μg/ml Amikacin) multiple times with shaking (100 rpm) for at least 30 minutes each wash. The biopsy tissue is weighed to determine the required volume of digestion solution to be made, then washed four times in disinfection solution (HBSS with 5 μg/ml Gentamicin) at room temperature for 2 minutes each wash. The tissue is cleaned of any fat or blood vessels with sterile scissors or scalpel on a sterile dish, then minced into fine pieces using a sterile blade. The minced tissue is transferred into a sterile conical tube and washed multiple times with disinfection solution by centrifugation at 600×g for 5 minutes each wash, then incubated with digest solution (1 mg/ml collagenase DE) for 1 hour, with shaking (100 rpm) at 37° C. The digested tissue is centrifuged at 600×g for 5 minutes then washed multiple times using disinfection solution and centrifugation at 600×g for 5 minutes each wash. Following the third wash, the pellet is subjected to a second enzymatic digest (1 mg/ml collagenase DE) for approximately 45 minutes, with shaking at 100 rpm at 37° C. Following the second digest, the remaining tissue is centrifuged at 600×g for 5 minutes, followed by multiple centrifugation washes with disinfection solution 5 minutes each at 600×g. A portion of the last wash is collected for sterility testing. The pellet is then resuspended by gentle pipetting in fresh smooth muscle growth media warmed to 37° C. The dissociated cells are plated into tissue culture vessels and incubated in a humidified incubator at 37° C. with 5% CO₂ for 4-5 days to facilitate cell attachment. After 4-5 days, fresh media is supplemented to all vessels every other day regularly.

### Cell Expansion and Harvest

### Expansion of Neural Progenitor Cells (NPC):

Neural progenitor cells are monitored for the formation of cell clusters under the microscope. When the cluster density reaches approximately 70%, cells are collected by centrifugation for 10 minutes at 2000×g and 15° C. The pellet is resuspended in 3-5 mL Accutase for 10 minutes, then neutralized using DMEM media. Cells are counted, centrifuged for 10 minutes at 200 g at 15° C., resuspended in fresh neural growth media, and re-plated and expanded to the desired number (˜ 5 million).

### Expansion of Smooth Muscle Cells (SMC):

When smooth muscle cells reach approximately 90% confluence, cells are harvested using TrypLE to release the cells from the plate and sub-cultured to expand to the desired number (˜10 million). Cells are counted and the percent of live cells is recorded.

### Cell Harvest:

Eight BioSphincter constructs are engineered per patient, necessitating a minimum of 4 million NPCs and 10 million SMCs. Since additional testing is required, when the expanded cell numbers exceed these values, the cells are harvested for manufacturing the final product (see, Table 1). During harvest, the spent media is collected to test for endotoxins and gram-positive organisms. A sample of the cells in the growth medium is also collected for measurement of cell count and viability, immunophenotyping (SMC) or immunocytochemistry (NPC), and detection of endotoxin, and gram-positive organisms before seeding.

TABLE 1
In-Process Specification and Acceptance Criteria for Cell
Number Required to Proceed to Seed of Collagen Hydrogels

| Cell | Cells per | Cells per 8 | Cells for | Total cells |
|---|---|---|---|---|
| type | BioSphincter | BioSphincters | QC testing | required |
| NPC | 0.5 × 10⁶ | 4 × 10⁶ | 2 × 10⁶ | 6 × 10⁶ |
| SMC | 1.25 × 10⁶ | 10 × 10⁶ | 3.0 × 10⁶ | 13 × 10⁶ |

### Preparation of Molds for the BioSphincters

Medical grade silicone is poured into 60 mm plates at 3-4 mm depth, centrifuged at 1000×g for 1 minute then allowed to cure at room temperature in horizontal position for one day. A custom-made 20 mm stainless steel biopsy punch is then used to make cylindrical posts of 3-4 mm height from the cured silicone. A single post is placed in the center of a 60 mm dish coated with partially cured medical grade silicone, which is then allowed to fully cure, creating one complete BioSphincter mold. The molds are sterilized using a validated ethylene oxide (ETO) cycle by Life Science Outsourcing (Brea, CA). Life Science Outsourcing has well-established procedures and extensive experience in ETO sterilization of medical components and shipping sterile components to and from its facility. ETO residual studies are also carried out and are described below. Following ETO sterilization, the molds are placed at room temperature for at least 7 days and then released for clinical use. Six 60 mm dishes each containing one silicone post mounted in the center are required for each patient.

### Ethylene Oxide (ETO) Residuals

Studies were performed to determine the level of ethylene oxide residuals in the BioSphincter molds following ETO sterilization. BioSphincter molds that had been ETO sterilized at LSO and returned to CELLF-BIO, LLC was subsequently shipped to WuXi AppTec for residuals testing by liquid extraction with USP purified water. This analysis demonstrated that 7 days post ETO sterilization, residual quantities of ETO and ethylene chlorohydrin (ECH) were below the allowable limits established in ISO 10993-7. Consequently, >7 days storage at room temperature post ETO sterilization has been designated as a threshold time for utilization of BioSphincter molds.

### Co-Culture of Cells

Four million NPCs are collected from the expanded culture and suspended in 24 mL of gel mixture consisting of medical-grade collagen, recombinant laminin, DMEM, and water (TABLE 2).

Simultaneously, a total of 10 million SMC are collected and pelleted from the expanded culture. The NPC suspension is gently mixed with SMC pellet by pipetting and a 4-mL volume of the cell mixture is dispensed by pipette around the central post in each of the 60 mm dishes. The dishes are then gently swirled to ensure complete coverage of the dish and the mixture is allowed to gel in the incubator at 37° C. with 7% CO₂ for at least 90 minutes for gelation.

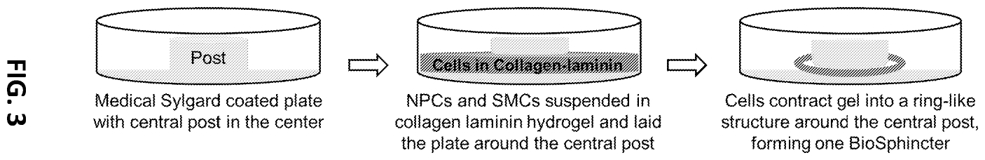

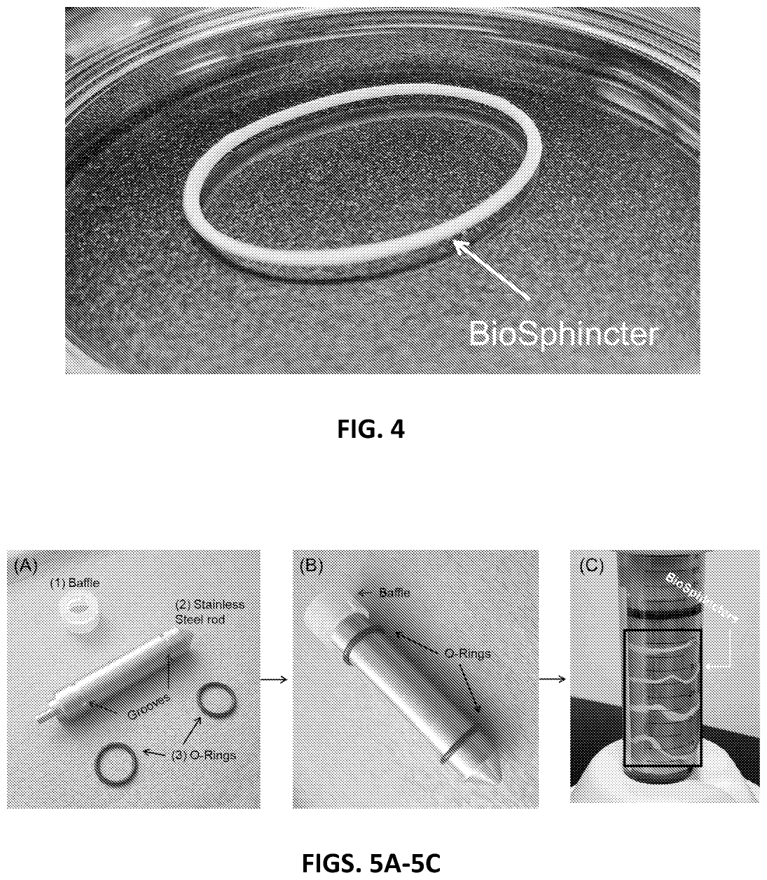

Following gelation, neural differentiation media is added to the dishes and transferred to the incubator at 37° C. with 5% CO₂. The media used to be changed every fourth day until day 12. Ultimately, the cell mixtures coalesce and form a ring structure around the central post within 48 hours. The BioSphincters used to mature by day 10-12 (FIG. 4).

Tissue viability, neural differentiation, innervation, and functionality are evaluated of the final product. The bioengineering process involves depositing cells into a hydrogel. The cells are allowed to concentrically align around a central post to form the BioSphincter construct. This maturation process takes 12 days to form an intrinsically innervated autologous cell BioSphincter.

During the bioengineering process, SMCs secrete a network of autologous human collagen, and the NPCs differentiate and form a functional neural network with concentrically aligned SMCs. The de novo production of autologous collagen replaces the initial bovine collagen in the hydrogel. Immunocytochemistry and qPCR analysis quantified that the seeded bovine collagen was significantly replaced (˜95%) by cell-synthesized autologous human collagen during remodeling. Therefore, the BioSphincter constructs used for implantation are considered to have a very low level of bovine collagen or recombinant human laminin remaining at the time of implant.

On the day of final product preparation, media is removed from the matured BioSphincters and tested for mycoplasma. Each BioSphincter is then washed 9 times in 10 mL HBSS. Each BioSphincter is carefully removed from around the cylindrical posts and allowed to remain in the dish. Randomly selected four BioSphincters are gently removed from the dish and transferred around the manufactured cylindrical shipper post. The shipper post is transferred to the transport vessel. One of the two remaining BioSphincter rings is homogenized into a final wash solution and tested for mycoplasma, sterility, endotoxin, and gram staining.

TABLE 2
Composition of Single- Layer Hydrogel Matrix
for Engineering the BioSphincters

| | Composition of single- layer | Per one |
|---|---|---|
| | hydrogel matrix | BioSphincter |
| 1 | SMCs (million) | 1.25 |
| 2 | NPCs (million) | 0.5 |
| 3 | Collagen (4 mg/ml) | 1000 ul |
| 4 | Collagen Buffer (μl) | 100 |
| 5 | 4X DMEM (μl) | 1000 |
| 6 | Laminin (μl) | 150 |
| 7 | Water (μl) | 1750 |
| | Total Volume per BioSphincter (μl) | 4000 |

### In-Process and Final Acceptance Testing for Product Release

The in-process testing is carried out at key points during the process and immediately prior to the bioengineering of BioSphincters. Release testing is performed on the final wash collected aseptically from each BioSphincter culture and one of the randomly selected BioSphincter. Release assays are performed according to approved SOPs, on validated and/or calibrated testing equipment. A written procedure for product release has been established.

### Transport to Clinical Site and Administration

A transport vessel design is described in FIGS. 5A-5C. A transport vessel consists of a 50 ml conical tube and a stainless-steel rod (4.35 inches in length and 0.75 inches in diameter) with O-Rings (Viton silicon, medical-grade silicon), and a baffle to stabilize the rod in the conical tube (FIGS. 5A, 5B). Four of the eight BioSphincters are randomly selected and placed onto the rod, followed by placement of O-rings at each end of the rod to prevent the BioSphincters from sliding off the rod (FIG. 5C). A baffle is positioned at the top and the rod containing the BioSphincters is placed within a 50-ml conical tube. Transport media is added to fill the tube. A sample of transport media is collected for sterility testing. The tube is capped, prepared for packaging into a qualified shipper that maintains a temperature of 15° C.-25° C., and transported to the clinical site for patient implantation within 48 hours of final product release. At the clinical site, the BioSphincters are removed aseptically from the shipping container in the operating room and implanted into the patient.

### Bioengineering of Human BioSphincters and Characterization:

After six weeks of culture of IAS-SMCs and enteric-NPCs, BioSphincters were bioengineered. Both isolated cells were mixed within bovine collagen-human recombinant laminin hydrogel and remodeling were studied during the bioengineering process. Tissue viability, neural differentiation, innervation, and functionality were evaluated in the final product. The bioengineering process involves depositing cells into a hydrogel. The cells are allowed to concentrically align around a central post to form the BioSphincter construct. This maturation process takes 12 days to form an intrinsically innervated autologous cell BioSphincter. The engineered BioSphincters were stained with specific antibodies directed against human collagen (type I and III) and bovine collagen (type I and III).

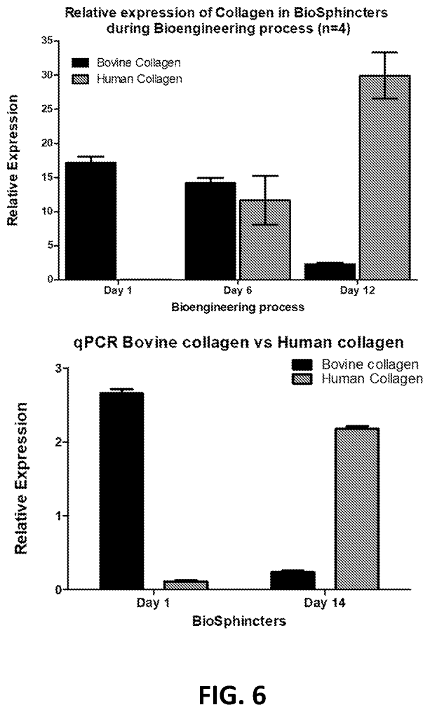

During the bioengineering process, SMCs secrete a network of autologous human collagen, and the NPCs differentiate and form a functional neural network with concentrically aligned SMCs. The de novo production of autologous collagen replaces the initial bovine collagen in the hydrogel. Immunocytochemistry and qPCR analysis quantified that the seeded bovine collagen was significantly replaced (˜95%) by cell-synthesized autologous human collagen during remodeling (FIG. 6). Therefore, the BioSphincter constructs used for implantation can have a very low level of bovine collagen or recombinant human laminin remaining at the time of implant.

### Route of Administration

Surgical implantation of the BioSphincters through a circumferential dissection around the anorectum.

### Example 2. Batch Analysis of BioSphincter

To assure the quality of the BioSphincter and the manufacturing process, in-process and release testing are performed.

In-process testing: In-process testing starts at the receipt of the biopsy samples and continues throughout the process, including isolation, culturing, and expansion of the cells, and manufacturing of the BioSphincters.

Release testing: Release testing occurs on the day of the Final Product collection. Endotoxin and Gram Staining results are available prior to the final product release. The final product is assessed for any microbial contamination by four different tests—1) Gram staining, 2) Endotoxins, 3) Mycoplasma, and 4) Sterility. All specifications for the tests have been determined using preclinical data. These four tests confirm the sterility of the final product.

Experiments were conducted using human biopsies. Representative results and analysis are described below.

### Results of Batch Analysis

The cell isolation and bioengineering of the BioSphincter were successfully completed. Release testing was performed on the final wash collected aseptically from each BioSphincter. The in-process testing (Table 3) was carried out at key points during the process and immediately prior to the bioengineering of BioSphincters. Release assays (Table 4) were performed on validated and/or calibrated testing equipment.

TABLE 3
In-Process Testing

| | | |
|---|---|---|
| Test | Process Intermediate | Specification |

In-Process Testing of Each Cell Type

| | | |
|---|---|---|
| Endotoxin | Spent Media | <20 EU/Implant |
| Gram Stain | Spent Media | Negative |
| Cell Growth | Cell Count | For Information and |
| Kinetics | | determination of |
| | | seeding density (NPC = |
| | | 6 × 10⁶; SMC = 13 × 10⁶) |

In-Process Cell Testing for Engineering
the BioSphincters
On Manufacturing Days

| | | |
|---|---|---|
| Cell Viability | Cells | >90% |
| Immunophenotype | Cells; final passage | ≥80% for given marker |
| | pre-manufacturing | αSMA (SMC) ≥80% |
| | BioSphincters | p75NTR ≥0% |
| Cell Count | Cells | NPC = 6 × 10⁶; |
| | | SMC = 13 × 10⁶ |
| Sterility | Cells | Negative |

TABLE 4
Release Testing

| | | |
|---|---|---|
| Test | Process Intermediate | Specification |

Collected and Tested 72 hours prior to the Shipment

| | | |
|---|---|---|
| Rapid Sterility | Spent Media, with one | Negative |
| Mycoplasma | BioSphincter | Negative |

Final Wash solution and Engineered BioSphincters
Collected and Tested at Time of Final Product

| | | |
|---|---|---|
| Gram Stain | Final Wash Supernatant, | Negative |
| | with one BioSphincter | |
| Endotoxin | Final Wash Supernatant, | <20 EU/Implant |
| | with one BioSphincter | |
| Physical | Visual Observation | Conforms to reference image |
| Integrity | | |
| Glucose/ | Spent Media | Metabolic detection of |
| Lactate | | glucose uptake. |
| | | Metabolic detection of |
| | | lactate production |
| Physiological | BioSphincter | The generation of |
| Functional | | Spontaneous Basal Tone |
| Analysis | | Contraction in response |
| | | to Potassium Chloride |
| | | Relaxation in response |
| | | to Electrical Field |
| | | Stimulation |
| Viability | BioSphincter | >70% Viability |

Collected at Time of Final Product and
Reported When Available (Post-Implantation)

| | | |
|---|---|---|
| Sterility - | Final Wash Supernatant, | Negative |
| 14 days | with one BioSphincter | |
| Mycoplasma | Final Wash Supernatant, | Negative |
| | with one BioSphincter | |

Bacteriostasis and Fungistasis (B/F) studies are completed on all media types prior to performing testing on clinical samples.

### Results of Manufacturing Runs

Clinical manufacturing runs were carried out to validate the ability of clinical manufacturing and representative results are shown in Table 5.

TABLE 5
Testing Methods and Results of Three Manufacturing Batches

| | | | |
|---|---|---|---|
| | | | Results (n = 3 |
| Test | Method | Specifications | manufacturing runs) |

Drug Substance Testing - Cells/Spent Media
Safety

| | | | |
|---|---|---|---|
| Endotoxin | Endosafe-PTS Charles | <20 EU/Implant | 0.005 ± 0.0005 |
| | River Laboratories | | |
| Gram Stain | Crystal violet stain | Negative | Negative |

Growth

| | | | |
|---|---|---|---|
| Cell Count | NucleoCounter NC-200 or | SMC: 1.25 million/ | Cell count achieved |
| | hemacytometer count | NPC: 0.5 million for | more than requirement |
| | | each BioSphincter | in each run. |
| Cell Viability | NucleoCounter NC-200 or | >70% | 89.28 ± 1.44 (SMCs) |
| | Trypan Blue exclusion | | 93.33 ± 0.80 (NPCs) |

Identity

| | | | |
|---|---|---|---|
| SMCs | BD FACSAria ™ III | αSMA (SMC) ≥80% | 98.33 ± 0.81% |
| NPCs | Immunocytochemistry | p75^{NTR} ≥80% | 99.6 ± 0.06% |

Drug Product Testing - Engineered BioSphincters (Final Product)
Sterility

| | | | |
|---|---|---|---|
| Aerobic | USP <71> | Negative | Negative |
| Anaerobic | USP <71> | Negative | Negative |
| Yeast/Fungal | USP <71> | Negative | Negative |
| Gram Stain | Crystal violet stain | Negative | Negative |

Purity

| | | | |
|---|---|---|---|
| Endotoxin | Endosafe-PTS Charles | <20 EU/Implant | 0.040 ± 0.024 |
| | River Laboratories | | |
| Mycoplasma | PCR | Negative | Negative |

Potency

| | | | |
|---|---|---|---|
| Physical | Visual Observation | Conforms to | Conforms with |
| Integrity | | reference | reference |
| Glucose/ | BioProfiler Basic 2 | Metabolic detection | Glucose uptake: 0.06 ± |
| Lactate | Chemical Analyzer | of glucose uptake | 0.007 g/BioSphincter |
| | | and lactate | Lactate Production: 0.13 ± |
| | | production | 0.004 g/BioSphincter |

### In-Process Testing Results

### Identification of Smooth Muscle Cells (SMCs)

Smooth muscle cells isolated from the IAS biopsy acquired a spindle-like morphology. Smooth muscle cells were isolated from three different IAS tissues according to CB-MBR-111-Isolation of Smooth Muscle Cells and expanded through several passages according to CB-MBR-113—Expansion for Smooth Muscle Cells. To identify and quantify the isolated cells, flow cytometry was performed on isolated, cultured smooth muscle cells after each isolation. Smooth muscle cells were incubated with anti-α-smooth muscle actin primary antibodies followed by the appropriate fluorescent secondary antibodies. Cells incubated with fluorescent secondary antibodies only were used as controls. Flow cytometry analysis demonstrated a high percentage of smooth muscle cells expressing muscle-specific antigens; α-smooth muscle actin, indicating the purity of the culture.

An average cell from three different tissue isolations was positive for α-smooth muscle actin was 80%. Therefore, the isolated cells qualified for bioengineering of the BioSphincters. Percent expressions are detailed in Table 6.

TABLE 6
Flow Cytometry of IAS-SMCs in Different Isolations

| # Manufacturing Run | IAS-SMCs | α-smooth muscle actin |
|---|---|---|
| 1 | Isolation 1 | 96.8 |
| 2 | Isolation 2 | 98.6 |
| 3 | Isolation 3 | 99.6 |

### Identification of Neural Progenitor Cells (NPCs)

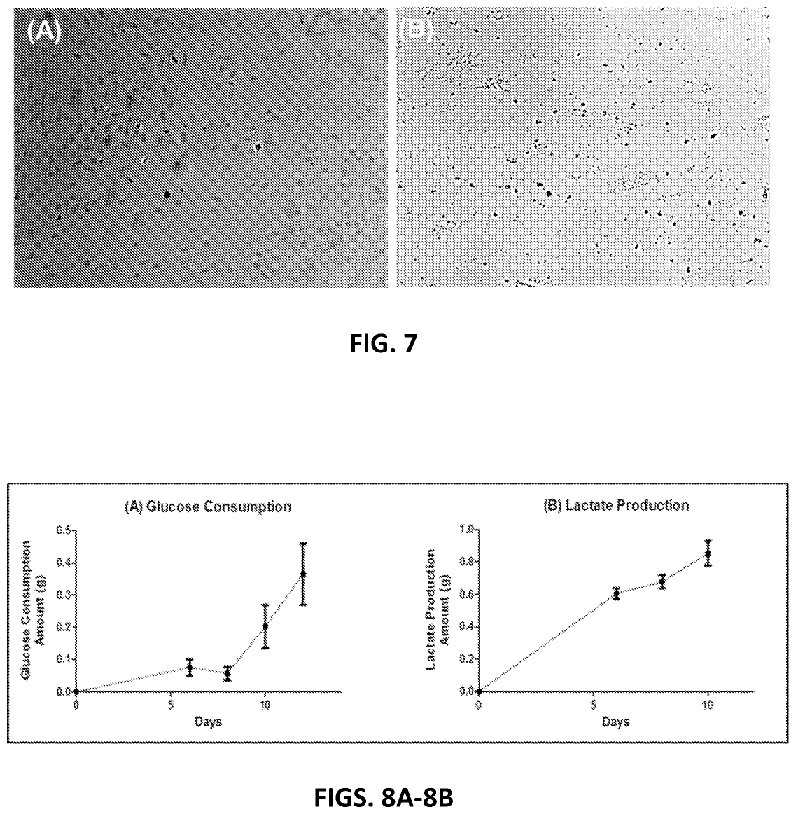

NPCs were isolated from multiple different SI tissues and expanded through several passages. Following each isolation, isolated cells were characterized via immunocytochemistry. The cells were stained with p75^{NTR} and smoothelin and Oct4 were used as a negative control. The Nikon Eclipse Ti inverted microscope was used for observation and image acquisition. The results are described in FIG. 7.

Immune Histomorphometric analysis of the cells (isolated from the small intestine) from each manufacturing batch (n=3) resulted in 99.6±0.06% stained positive for p75^{NTR} confirming the identity of isolated cells as NPCs. (FIG. 7). Therefore, the isolated cells qualified for bioengineering of the BioSphincters. These expressions were consistent for up to five generations and reduced afterward. The cells were negative for the pluripotency markers Oct4 and smoothelin.

### STAT Gram Staining

Gram stain tests are carried out in CELLF-BIO, LLC using the standard kits (FDA 510(K) approved) based on crystal violet stain. Gram staining is carried out on both types of cells. The test results were negative and met the acceptance criteria.

### Endotoxin Testing

Endotoxin testing was carried out for endotoxins Endosafe from Charles River Laboratories. The Endosafe® nexgen-PTS™ is a rapid, calibrated, validated equipment, point-of-use handheld spectrophotometer that uses USP/BET-compliant disposable cartridges for accurate, convenient, and real-time endotoxin testing, glucan concentration determination, and Gram identification. This equipment is 21 CFR Part 11 compliant. Endotoxin testing was carried out on spent media of both the cells and the results came negative and in the acceptance range (<20 EU/Implant).

### Release Testing Results

### Physical Integrity

The final product is a BioSphincter, which is a ring structure with a 20-mm diameter central open lumen (FIG. 4).

One of the BioSphincter (from eight bioengineered BioSphincters) is randomly selected and dimensions were measured with the help of calibrated vernier calipers. The internal diameter, height, and thickness of the BioSphincter were measured. The internal diameter needs to be 20 mm to release the product to the clinical site. With help of all the measurements, surface area and surface volume were also calculated for the record.

The surface area of the BioSphincters averaged 140.90±0.85 mm² and volume-averaged 153.87±0.62 mm³. The thickness of the BioSphincter averaged 1.92±0.01 mm. The height of the BioSphincter averaged 2.44±0.01 mm. Values are means±SEM; N=3.

### Glucose Lactate Assay

To confirm the cell viability and metabolic activity, a glucose lactate assay was performed. Spent media was collected at the time of media changes during the culture process for BioSphincters, beginning at day six of post-bioengineering up to day 12. The amount of glucose and lactate was measured in the collected media in order to assess cellular metabolism. The results are described in FIGS. 8A-8B. As shown in FIGS. 8A-8B, these assays indicate that the levels of glucose consumption and lactate production significantly increase. The average glucose uptake and lactose production for BioSphincters (n=3) were 0.036±0.007 g/BioSphincter and 0.13±0.004 g/BioSphincter respectively.

### STAT Gram Staining

Gram stain tests are carried out in CELLF-BIO, LLC using the standard kits (FDA 510 (K) approved) based on crystal violet stain. The testing was carried out on one of the randomly selected BioSphincter homogenized in the final wash solution and the results were negative and met the acceptance criteria.

The testing was carried out on one of the randomly selected BioSphincter homogenized in the final wash solution and the results came negative and met the acceptance criteria.

### Endotoxin Testing

Endotoxin testing was carried out for endotoxins Endosafe from Charles River Laboratories. The Endosafe® nexgen-PTS™ is a rapid, calibrated, validated equipment, point-of-use handheld spectrophotometer that uses USP/BET-compliant disposable cartridges for accurate, convenient, and real-time endotoxin testing, glucan concentration determination, and Gram identification. This equipment is 21 CFR Part 11 compliant. The testing was carried out on one of the randomly selected BioSphincter homogenized in the final wash solution and the results were negative and in the acceptance range (<20 EU/Implant).

### Sterility Safety

Sterility testing includes testing for aerobic, anaerobic, and yeast/fungal sterility cultures. The testing was carried out on one of the randomly selected BioSphincter homogenized in the final wash solution and the results were negative.

### Mycoplasma Testing (USP 71)

Mycoplasma testing includes ‘Rapid Mycoplasma Detection by DNA Amplification for Autologous Cell Therapies. Interference for Rapid Mycoplasma Detection by DNA Amplification for Autologous Cell Therapies was also carried out. The testing was carried out on one of the randomly selected BioSphincter homogenized in the final wash solution and the results were negative.

### Example 3. Comparative In Vivo Studies

Rabbits and non-human primates (NHPs) are used in pre-clinical studies (Bohl et al., 2017; Dadhich et al., 2019; Zakhem et al., 2019). The animals are randomly divided into three groups (Table 7): (1) Non-treated group (control) undergoing a survival surgery for internal anal hemi-sphincterectomy to induce passive FI without any further treatments; (2) The treated group undergoing a survival surgery for internal anal hemi-sphincterectomy to cause passive FI followed by a second survival surgery (6 to 8 weeks later) for implantation of BioSphincters; (3) The sham group undergoing a survival surgery for internal anal hemi-sphincterectomy to cause passive FI followed by a second survival surgery (6 to 8 weeks later) where the surgical site was re-accessed without implantation of BioSphincters.

### Surgeries

All surgical procedures involving either the rabbits (n=26) or non-human primates (n=10) are performed under good laboratory practices (GLP) and strict aseptic conditions following the species-specific surgical guidelines set forth by IACUC.

Small intestinal biopsy: Laparotomy for small intestinal biopsy is performed on all animals for cell isolation. An antimesenteric biopsy is taken and the biopsy is used to isolate neuro progenitor cells.

Internal anal hemi-sphincterectomy: Internal anal hemi-sphincterectomy is performed on all animals to develop the model of passive FI. The IAS is identified and dissected from the submucosal plane and the overlying external anal sphincter. IAS tissue is then immediately transferred for cell isolation.

Implantation of engineered autologous BioSphincters: Six to eight weeks following the first surgery (intestinal biopsy and sphincterectomy), animals in the treated group and sham are scheduled for a second survival surgery. A circumferential incision is made around the anocutaneous junction of the anal canal. The inter-sphincteric plane is identified and dissected for approximately 1-cm proximally. In the treated group, four engineered autologous BioSphincters are placed and stacked circumferentially next to each as full muscle rings in the inter-sphincteric space. In the sham group, the dissection plane is then closed without any implantation.

TABLE 7
Study Design for the Pre-clinical Study

| | | | | | Manometry | 4-6 weeks | | | | |
|---|---|---|---|---|---|---|---|---|---|---|
| | | | | Sphincter- | post | post | | | | |
| | Rabbits - | NHPs - | Baseline | ectomy to | sphincter- | sphincter- | 1 | 3 | 6 | 12 |
| | No. | No. | manometry | Induce FI | ectomy | ectomy | month | months | months | months |
| Non- | 11 | 2 | ✓ | ✓ | ✓ | No treatment | Manometry | Manometry | Manometry | Manometry |
| treated | | | | | | | post- | post | post | post |
| group | | | | | | | sphincter- | sphincter- | sphincter- | sphincter- |
| | | | | | | | ectomy | ectomy | ectomy | ectomy |
| Treated | 10 | 6 | ✓ | ✓ | ✓ | Implant | Manometry | Manometry | Manometry | Manometry |
| group | | | | | | BioSphincters | post- | post- | post- | post- |
| | | | | | | | implant | implant | implant | implant |
| Sham | 5 | 2 | ✓ | ✓ | ✓ | Sham surgery | Manometry | Manometry | Manometry | Manometry |
| group | | | | | | | post- | post- | post- | post- |
| | | | | | | | sham | sham | sham | sham |

Hemi-sphincterectomy of the IAS to induce FI. Cells are used to engineer BioSphincters in vitro and implanted into the treated group. Anorectal Manometry is performed at regular intervals together with the observation of fecal hygiene to assess the status of the animals' welfare. A total of 26 rabbits and 10 NHPs are included in the study.

### Cell Isolation and Bioengineering of BioSphincters

Enteric-NPCs are harvested from small intestine biopsies. IAS biopsy is processed to isolate SMCs. After six weeks of the culture of enteric-NPCs and of IAS-SMCs, autologous BioSphincters are bioengineered for implantation (Bohl et al., 2017; Dadhich et al., 2019; Zakhem et al., 2019). The BioSphincters are optimized for individual species, for rabbits the lumen diameter of BioSphincters is 8 mm, whereas for NHPs the lumen diameter is 12 mm. BioSphincters take 10 days for neural differentiation, maturation, and gaining sphincteric functionality.

Quality control of the autologous intrinsically innervated IAS is performed through real-time force generation to evaluate the physiological functionality of the BioSphincters. All constructs are able to generate basal tone, a characteristic of the IAS.

### In Vivo Efficacy Study

Animals in the non-treated group and sham group continue to exhibit a lack of fecal hygiene and maintained a reduced anal basal pressure and rectoanal inhibitory reflex (RAIR) throughout the study. Animals in the treated group demonstrate reinstatement of fecal hygiene, normal anal basal pressure, and RAIR post-implantation of the BioSphincters. After euthanasia at the pre-determined endpoints, physiological testing show that the harvested IAS implant is viable and maintain the physiological characteristics of IAS. In the histological analysis, tagged neural cells in implanted BioSphincters display neuronal interaction and synaptic junctions with host neural cells.

Statistical analysis: Analysis of basal tone and RAIR is analyzed using BioVIEW software (Sandhill Scientific, Littleton, CO). One-way ANOVA followed by Tukey's test is used to compare the basal tone, and RAIR values regularly recorded at baseline and at the indicated time points of 3, 6, and 12 months in the non-treated group, treated group, and sham group.

### SUMMARY

FI is induced in the rabbit and NHPs by producing a hemi-sphincterectomy (180° resection of the anterior IAS sphincter). Changes in fecal control are evident in all animals based on fecal staining of the perineum and scatter of fecal material throughout the cage. There is a significant decrease in anal basal pressure and RAIR compared to baseline (no surgery), indicating the validity of the incontinent model.

Implantation of four BioSphincters following the hemi-sphincterectomy can restore both anal basal pressure and RAIR (significantly higher than values in the non-treated group). There can be an improvement in defecatory activity. Compared to non-treated and sham groups, manometry readings in the treated group can confirm that the BioSphincters are viable and functional in vivo.

### Example 4. Manufacturing Process Development for Clinical Size/Medical Grade BioSphincters

### Comparing Preclinical Non-Clinical Processing and Human Biopsy Processing

TABLE 8
Preclinical Processing vs. Human Processing

| | | |
|---|---|---|
| | Proposed Human | |
| Nonclinical Processing | Processing | Rationale/Justification |

Isolation and culture of smooth muscle cells

| | | |
|---|---|---|
| Digestion mix: type 2 | Digestion mix: | In human biopsy processing, |
| collagenase + DNase | Collagenase DE400 | enzymes and most of the |
| Processing buffer: | Processing buffer: | reagents are switched to xeno- |
| HBSS with phenol | HBSS (phenol red- | free, except smooth muscle |
| red + Gentamicin + | free) + Gentamicin | growth media where FBS was |
| antibiotic/antimycotic | SMC growth media: | used from FDA acceptable |
| SMC growth media: | Promocell Media + | stocks. |
| DMEM high glucose + | EGF + bFGF + | (Refer Tech Study IAS- |
| 10% FBS + L- | Insulin + | CellfBio-TS01 Isolation of IAS |
| Glutamine + | Gentamicin + | Smooth Muscle Cells - Testing |
| antibiotic/antimycotic | 2% FBS | 2 different methods) |

Isolation and culture of neural progenitor cells

| | | |
|---|---|---|
| Digestion mix: type 2 | Digestion mix: | In human biopsy processing, |
| collagenase + | Collagenase HA + | enzymes and reagents are |
| Dispase + DNase | BP protease | switched to xeno-free. |
| Processing buffer: | Processing buffer: | (Refer Tech Study IAS- |
| HBSS with phenol | HBSS (phenol red- | CellfBio-TS02 Isolation |
| red + Gentamicin + | free) + Gentamicin | of SI neural progenitor |
| antibiotic/antimycotic | Neural growth media: | cells - Testing 2 different |
| Neural growth media: | Neurobasal + N2 | methods) |
| Neurobasal + N2 | supplement + EGF + | |
| supplement + EGF + | bFGF + L-Glutamine + | |
| FGF + L-Glutamine + | Gentamicin | |
| antibiotic/antimycotic | | |

Engineering BioSphincters

| | | |
|---|---|---|
| Sylgard 184 | Medical grade Silastic | The size of human IAS is |
| Plate size: 35 mm | silicone | larger than rabbit IAS. The |
| Post size: 8 mm | Plate size: 60 mm | dimensions of human |
| Smooth muscle cell | Post size: 20 mm | BioSphincters were |
| density/BioSphincter: | Smooth muscle cell | proportionally increased |
| 500,000 | density/BioSphincter: | compared to the rabbit |
| Neural progenitor | 1.2 million | BioSphincters. |
| cell density/ | Neural progenitor cell | Reagents were switched to |
| BioSphincter: 200,000 | density/BioSphincter: | medical grade, USP grade, |
| Rat tail collagen type | 500,000 | and Clinical/CTG Grade. |
| 1-1.6 mg/ml | Collagen I medical | (Refer Tech Study IAS- |
| Mouse laminin | grade - 4 mg/ml | Cellfbio-10 Scale-up |
| FBS | Recombinant laminin | engineering of BioSphincters |
| 4X DMEM | Neutralizing buffer | using clinical-grade reagents) |
| Water | 4X DMEM (phenol | |
| Neural differentiation | red-free) | |
| media: Neurobasal-A + | Water | |
| B27 supplement + | Neural differentiation | |
| 1% FBS + | media: Neurobasal-A + | |
| antibiotic/antimycotic | B27 supplement + | |
| | 1% FBS + Gentamicin | |

### Isolation of Internal Anal Sphincter Smooth Muscle Cells-Evaluation of Two Methods

### Objective

This Example studied the isolation of IAS Smooth muscle cells (SMCs) using animal-free, clinical-grade reagents (Experimental) compared to the traditional non-clinical isolation method that uses animal-sourced reagents (Control).

The established nonclinical protocols for isolation of smooth muscle cells from IAS tissue involve the use of animal-sourced reagents (collagenase type 2, trypsin) and seeded in culture media prepared with DMEM with 10% FBS (control). The goal of this study is to evaluate the use of animal-free enzymes DE Collagenase 40 for the isolation of cells (experiment) following culture in promo cell media.
The digestive reagents tested in this study will be animal-free and clinical grade.
The growth, morphology, and identity of the cells will be evaluated under control and experimental conditions.

### Procedure

Biopsy transport
Prepared a sterile specimen container that contains HBSS+50 μg/ml gentamicin.
Biopsy tissue was received from the OR and the specimen was delivered for processing.

### Cell Isolation

The transport container was cleaned into the BSC.
IAS tissue was gently removed from the container and placed in a sterile tube.
IAS tissue was incubated in 50 mL Antibiotic Solution for 10 minutes and repeated 2 more times.
Total 1 mg/ml enzymes were reconstituted for each group.
   For the experimental group, DE collagenase 400 (011-1030-VitaCyte) was reconstituted at 1 mg/ml concentration.
   For control group, 1 mg/ml Collagenase type 2 (Cat #LS004177).
IAS Biopsy was harvested, weighed, and was split into 2 equal pieces by weight.
Each biopsy piece was placed in a separate tube and labeled appropriately:
   Control: Biopsy processed following Nonclinical protocol.
   Experimental: Biopsy processed following clinical protocol CB-MBR-111
Biopsy pieces were washed by rinsing in approximately 40 mL disinfection solution three times
Washes were repeated thrice using new tubes for each wash.
After the washes, the biopsy piece was placed onto Petri dish and was minced into small pieces approximately <1 mm.
Minced tissue was transferred into the conical tube and washed three times with 40 mL disinfection solution 600×g for 5 minutes.
The tissue was transferred into the appropriate digest mixture.
Flasks containing minced tissue and digest mix were secured and incubated at 37° C. in a shaking incubator with shaking at 100 rpm for 60 minutes, then centrifuged at 600×g for 5 minutes.
The supernatant was carefully discarded, and the pellet was washed 3 times with centrifugation at 600×g for 5 minutes after each wash.
The pellets were digested again as described above, for 45-60 minutes and the tissue digest was centrifuged at 600×g for 5 minutes.
The supernatant was carefully discarded, and the pellet was washed 3 times with centrifugation at 600×g for 5 minutes after each wash.
Following the last wash, the supernatant was discarded, and 5 ml growth media was added to each pellet as indicated:
   For the control tissue, DMEM with 10% FBS
   For the experimental tissue, Smooth muscle growth media-Clinical
The digested tissue was mixed by pipetting up and down to dissociate from each other, plated into T-75 flasks, and placed into a 37° C. humidified incubator with 5% CO₂.

### Cell Characterization

Cells were visually examined under the microscope for cell attachment and morphology.
Cell counted using Nucleocounter; plated in triplicate and cultured for 7 days and proliferation was compared between two different methods.
For identity purposes, immunocytochemistry was performed to detect the expression of Smoothelin, a contractility protein present in SMCs.
At various passages, SMCs were also assessed for smoothelin expression by qPCR.

### Data Summary and Conclusion

Isolation of cells from internal anal sphincter tissue was conducted with two different enzymatic digest and culture conditions:
   Collagenase DE400; Smooth Muscle Cell Media-Clinical
   Collagenase type 2; DMEM/10% FBS
Cells isolated under each condition exhibited characteristics of smooth muscle cells:
   The cell isolation from both methods resulted in similar cell yield in spindle-like morphology, there was no significant difference (p<0.05) in the proliferation rate of the cells isolated by the two different methods.
   qPCR demonstrated a similar smoothelin expression using both the methods without significant difference (p=0.0015).
   An average cell from three different tissue isolations was positive for α-smooth muscle actin was 98.3% percent expressions for experimental and 96.8 percent expressions for Control.
Cells were isolated from internal anal sphincter tissue by two different methods. The established nonclinical method for isolation of smooth muscle cells from IAS tissue involves the use of animal-sourced reagents (collagenase type 2, trypsin) and seeded in culture media prepared with DMEM with 10% FBS (control).
When compared this method by substituting the digestive enzyme with Collagenase DE400 and culture media with Clinical grade cell growth media, resulted in a similar yield of SMCs. The isolated cells with the new method were similar in proliferation and smoothelin expression. The data showed that the cells isolated from these procedures performed similarly in culture, supporting the use of clinical-grade reagents for isolation and cell media in expansion for clinical use.

### Isolation of SI Neural Progenitor Cells—Testing 2 Different Methods

### Objective

T study the isolation of small intestinal Neural Progenitor Cells (NPCs) using animal-free, clinical-grade reagents (Experimental) compared to the traditional non-clinical isolation method that uses animal-sourced reagents (Control).

Currently, the established protocols for the isolation of NPCs involve the use of animal-derived enzymes (collagenase type 2, dispase II). The goal of this study is to evaluate the use of animal-free enzymes/reagents for the isolation and expansion of NPCs.

The isolation of NPCs will be compared for morphology, phenotype, and gene expression as described in the acceptance criteria.

### Procedure

### Biopsy Transport:

Prepared a sterile specimen container that contained HBSS+50 μg/ml gentamicin.
Small Intestine (SI) biopsy tissue was received from the OR and the specimen was delivered for processing.

### Cell Isolation:

The transport container with biopsy tissue was cleaned into the BSC.
The SI biopsy tissue was gently taken out from the container and transferred into a tube containing 50 mL Antibiotic Solution for 10 minutes and repeated 2 more times.
The enzymes for digestion were reconstituted: Collagenase HA and BP protease (Collagenase HA source is VitaCyte and BP protease from VitaCyte according to the protocol in CB-PBR-101 for the Experimental group
Collagenase type 2/dispase/DNase according to PR-SOP-057 for the Control group.
The tissue was divided into two equal pieces by weight.
Each tissue sample was added into a separate sterile tube and labeled appropriately as control and experimental.
The tissues were washed by rinsing in approximately 10 mL disinfection solution three times
Tissue sections were then placed on a tissue culture plate and minced into pieces approximately 1 mm or smaller
The minced tissues were transferred into sterile tubes and washed:
Washing was repeated three times with gentle agitation followed by centrifugation at 600×g for 5 minutes using either 40 mL of Disinfection Solution for the Experimental group or 40 mL of HBSS for the Control group
Once washed the minced tissue was mixed with appropriate digest and incubated at 37° C. in a shaking incubator at 100 rpm for approximately 60 minutes.
Digested samples were centrifuged at 400×g for 5 minutes.
The supernatant was collected without disturbing the pellet and passed through a 70 μm strainer into a new tube which was incubated at 4° C. (on ice).
The remaining tissue pellets from each group were digested again for 45-60 minutes using the appropriate digest mix then centrifuged at 400×g for 5 minutes and the supernatant was collected and passed through a 70 μm strainer into the new sterile 50 ml centrifuge tube while discarding the pellet.
The previously collected 70 μm passed supernatant and present supernatants were combined and centrifuged at 1800-2000×g for 10 minutes
The cell pellet was washed 3 times by centrifuging at 1800-2000×g for 10 minutes using appropriate wash solutions.
Following the washes, the wash supernatant was discarded, and each condition cell pellet was suspended in NPC growth media.
The cell suspensions were passed through 40 μm strainers into a new tube and the cell filtrate was plated into an appropriate number of non-tissue culture-treated flasks.
The isolated cells in experimental groups were cultured in non-tissue culture flasks containing clinical-grade neural growth media: according to CB-MBR-102.
The cells isolated in the control group were cultured in non-tissue culture flasks containing non-clinical grade neural growth media

### Cell Characterization

Cell viability assay was carried out on Days 1, 7, and 15.
The viability assay was performed to measure cell viability for each construct. Measurement of cell metabolic activity is one of the common methods to assess the viability of cells.
Isolated cells were stained for p75 which is a characteristic of neural crest-derived cells. The semi-quantitative histomorphometry analysis was carried out using ImageJ software (NIH, USA).
At various passages, NPCs were also assessed for p75 expression by qPCR.

### Data Summary and Conclusion

Isolation of cells from small intestine biopsies was conducted with two different enzymatic digest and culture conditions:
   Collagenase HA and BP protease; Neural progenitor growth media-Clinical
   Control-Collagenase type 2/dispase/DNase; Neural progenitor growth media-non-Clinical
Cells isolated under each condition exhibited characteristics of neural progenitor cells:
   The cell isolation from both methods resulted in similar cell yield, there was no significant difference (p<0.05) in the proliferation rate of the cells isolated by the two different methods.
   qPCR demonstrated similar smoothelin expression using both the methods without significant difference (p<0.05).
   In the immunocytochemical analysis, cells from both the isolation exhibited similar expression for p75^{NTR} (neural crest) marker.
Cells were isolated from internal anal sphincter tissue by two different methods. The established nonclinical method for isolation of neural progenitor cells from small intestine tissue involves the use of animal-sourced reagents (Collagenase type 2/dispase/DNase) and seeded in culture media prepared with DMEM with 10% FBS (control).
When compared this method by substituting the digestive enzyme with Collagenase HA and BP protease and culture media with Clinical grade cell growth media, resulted in a similar yield of NPCs. The isolated cells with the new method were similar in proliferation and p75^{NTR} expression. The data suggest that the cells isolated from these procedures performed similarly in culture, supporting the use of clinical-grade reagents for isolation and cell media in expansion for clinical use.

### Objective

To scale up the engineering process of the BioSphincters from a pre-clinical animal size to clinical human-size using medical-grade reagents.

The IAS BioSphincters are successfully bioengineered and implanted of 8- and 12-mm internal diameter (ID) in rabbits and non-human primate models, respectively. The BioSphincters contain rat tail collagen, mouse laminin, and FBS. In this study, we bioengineer clinical size/medical grade BioSphincters using clinical-grade reagents.

The objective of the study is to study the bioengineering of BioSphincters' clinical-grade laminin and collagen (Experimental) compared to the traditional non-clinical bioengineering of BioSphincters which uses rat tail collagen, mouse laminin, and FBS (Control).

The BioSphincters will be scaled up to clinical size constructs to 20 mm ID. The gel mixture will contain clinical-grade laminin and collagen without FBS. Constructs will be tested for structure, functionality, and differentiation capacity (qPCR).

### Procedure

SMC and NPC cells used in this study were isolated from human IAS and human small intestines, respectively. Cells were isolated following the methods described in the foregoing sections.

### BioSphincter Preparation Using Nonclinical Process and Reagents:

Collected NPC (500,000 per construct) and SMC (1.25 million per construct).
Re-suspended the NPC in the gel (rat tail collagen type 1, mouse laminin, 4× DMEM, water) and poured it on a 60 mm Sylgard-coated plate with a 20 mm post in the center of the plate.
Allowed the gel to polymerize at 37° C. for at least 15-20 minutes
Resuspended the SMC in the same gel composition (except for laminin) and poured it on top of the first layer of gel.
Allowed it to gel at 37° C. for another at least 15-20 minutes.
Added the non-clinical-neural differentiation media to the plate.

### BioSphincter Preparation Using Clinical Grade Reagents:

Collected NPC (500,000 per construct) and SMC (1.25 million per construct).
Re-suspended the NPC in the gel (clinical grade collagen, buffer, recombinant laminin, 4×DMEM, water) and mixed it with the SMCs in the same gel composition, and poured it on a 60 mm Sylgard-coated plate with a 20 mm post in the center of the plate.
Allowed it to gel at 37° C. for another at least 90 minutes.
Added the clinical-grade-neural differentiation media.

### Characterization of BioSphincters

Immunophenotyping through Immunocytochemistry: These studies were performed following the standardized SOP. The SOPs are standardized according to the manufacturer's instructions (Immunocytochemistry and immunofluorescence protocol, Abcam, USA).

qPCR: qPCR was performed in order to measure the expression of smoothelin and βlll tubulin.

Functional analysis: Engineered BioSphincters were analyzed for physiological functionality. The BioSphincter was hooked in a horizontal tissue bath (Harvard Apparatus) and data were acquired using LabChart-7 software (AD Instruments). The spontaneous basal tone was measured. Effect of the excitatory (Acetylcholine (ACh; 40 μmol/l) and relaxant (electrical field stimulation; EFS; 5 Hz, 0.5 ms) stimulants were evaluated.

### Data Summary and Conclusion

BioSphincters scaled up to clinical size: Scaled up BioSphincters of internal diameter (ID) of 20 mm were successfully engineered using the medical-grade collagen and clinical-grade laminin.
qPCR studies concluded similar βlll tubulin and smoothelin expression of BioSphincters engineered in two different methods.
Physiological Functional studies indicated similar responses to different excitatory and inhibitory stimulants (without any significant difference) to the BioSphincters engineered with different types of collagens.
Engineering BioSphincters using animal-free reagents was feasible. Successfully scaled up to clinical sizes.
Scaling up BioSphincters from 0.8 cm ID to 2 cm ID was successful. BioSphincters were engineered using medical/clinical-grade materials and performed similarly to control BioSphincters which were engineered using animal-sourced materials. Medical grade collagen, recombinant laminin, and omitting FBS from the recipe of the gel did not affect the formation of the constructs.

### Example 5. Physicochemical Properties of the BioSphincters

Three clinical manufacturing runs were carried out to validate the ability of clinical manufacturing and the results are as follows:

### Structure and Dimensions

A BioSphincter that has a ring structure with a central lumen was made. The internal diameter of the BioSphincter was 20 mm. The surface area of the BioSphincters averaged 140.90±0.85 mm² and the volume averaged 153.87±0.62 mm³. The thickness of the BioSphincter averaged 1.92±0.01 mm. The height of the BioSphincter averaged 2.44±0.01 mm. Values are means±SEM; N=3.

### Physiological Functionality

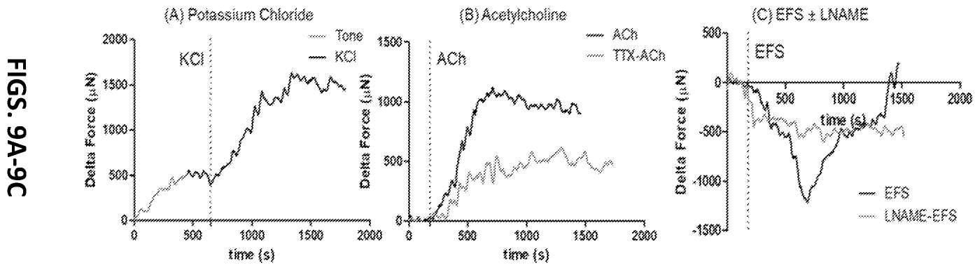

BioSphincters were analyzed for the functionality of both SMC and NPC components on day 12 post-bioengineering by measuring real-time force generation on an isometric force transducer. Briefly, BioSphincters were incubated in fresh 37° C. HEPES buffer to establish a baseline for basal tone and then treated with 60-mM KCl to induce depolarization of the smooth muscle membrane. As shown in FIG. 9A, KCl-induced depolarization results in smooth muscle contraction (average 1081±14 μN; n=5). Smooth muscle contraction in response to membrane depolarization reflects the maintenance of functional voltage-dependent Ca²⁺ channels in the smooth muscle cells within the BioSphincters.

BioSphincters were also tested for contractility in response to the major excitatory neurotransmitter in the gut, Acetylcholine (Ach). As shown in FIG. 9B, treatment with exogenous Ach (10 μM) results in contraction of the BioSphincters with an average peak contraction of 949.4±28.5 μN (n=5). To distinguish between the muscle and neural contribution of the response, BioSphincters were washed, incubated in fresh buffer, and treated with tetrodotoxin (TTX), an inhibitor of voltage-gated Na+ nerve channels. As shown in FIG. 9B-red trace, treatment with Ach (10 μM) under this condition induced a lower level of contraction that averaged 392.8 #15.9 μN (n=5). These results demonstrate that both smooth muscle and neural components of the BioSphincters responded to Ach.

The neural component of the BioSphincter system was also tested by using electrical field stimulation (EFS). EFS (parameters: 5 Hz, 0.5 ms) was applied to the BioSphincter with parallel platinum plate electrodes in the organ bath and resulted in a relaxation response with maximal relaxation averaging −958±67.7 μN (n=5) (FIG. 9C). Relaxation was abolished by TTX pretreatment (−337.2±6.0 μN) (n=5). To further characterize the relaxation, BioSphincters were also pre-treated with nitric oxide synthase inhibitor, N(ω)-nitro-L-arginine methyl ester (L-NAME; 300 μM, nNOS inhibitor) followed by EFS. Relaxation was partially inhibited at −512.2±17.1 μN (n=5) (˜50% inhibition), indicating the presence of functional nitric oxide neural population that contributed to the relaxation response (FIG. 9C-green trace). Together, these results demonstrate that the NPC populating in the BioSphincter differentiate into functional neurons by day 12 post-bioengineering. Results are summarized in Table 9.

TABLE 9
Summary of Physiological Functionality
Physiological functional assay prior to shipment of the final product (n = 5)

| | | Tested Parameters | | | | | | |
|---|---|---|---|---|---|---|---|---|
| Clinical | Measurement | | | | | | | TTX- |
| Engg. Run | (Unit) | Tone | KCl | Ach | TTX-Ach | EFS | TTX-EFS | Lname |
| Avg. of 5 | Force ± SD | 261.20 ± | 1081.0 ± | 949.40 ± | 392.8 ± | −958 ± | −337.2 ± | −512.2 ± |
| Runs | (μN) | 29 | 14.3 | 28.5 | 15.9 | 67.7 | 6.0 | 17.1 |

These results demonstrated that both the neuronal and muscular components were functional in the BioSphincters.

### Example 6. Biological Properties of the BioSphincters

### Glucose/Lactate Assay

The Glucose/lactate assay was performed as described in the foregoing paragraphs. Spent media is collected at the time of media changes during the culture process for BioSphincters, beginning at day six of post-bioengineering up to day 12. The amount of glucose and lactate was measured in the collected media in order to assess cellular metabolism. The results are described in FIGS. 8A-8B. As shown in FIGS. 8A-8B, these assays indicate that the levels of glucose consumption and lactate production significantly increase. The average glucose uptake and lactose production for BioSphincters (n=3) were 0.036±0.007 g/BioSphincter and 0.13±0.006 g/BioSphincter, respectively.

### Viability Assay

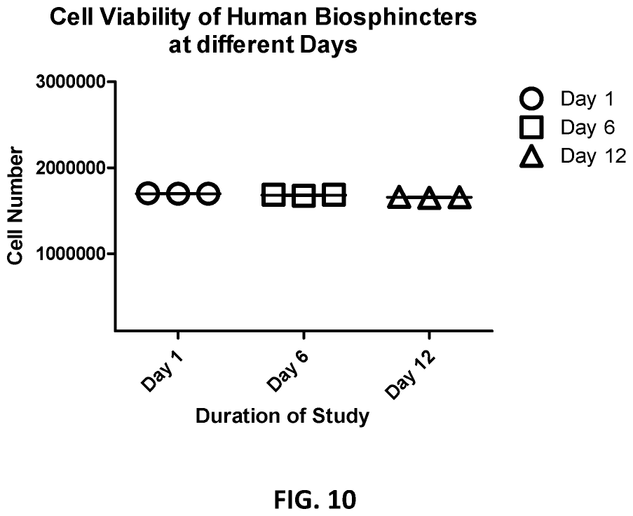

The viability of both NPC and SMC in the BioSphincter was followed from day 1 up to day 12 post-bioengineering. BioSphincters were harvested on day 1, day 6 and day 12 for MTT (3-[4, 5-dimethylthiazol-2-yl]-2, 5 diphenyl tetrazolium bromide) dye reduction assay. The MTT assay was carried out with cell-seeded scaffolds at different time intervals. The absorbance was considered directly proportional to the number of live, metabolically active, and growing cells. Cells at day 0 of engineering were also harvested and used for standard curve generation. BioSphincters were homogenized and incubated with 0.5 mg/mL MTT solution for 4 hours at 37° C. During this incubation, the MTT reagent is reduced by the cells to purple formazan crystals that can be detected spectrophotometrically at 570 nm. A standard curve for cell number was generated and used to extrapolate the number of cells in the BioSphincters at various time points. Cell viability did not significantly change from day 1 to day 12, indicating maintenance of the integrity of the cells (FIG. 10). The total number of viable cells dropped slightly by 3% from day 1 to day 12.

### Immunocytochemistry

On day 12 post-bioengineering, BioSphincters were fixed and processed for histological analysis. Immunofluorescence studies were performed. Slides were de-paraffinized and hydrated to water. Slides were blocked using serum followed by permeabilization. Immunocytochemical analysis was performed with an antibody specific for α-smooth muscle actin, conjugated with a green fluorescent protein. Fluorescence microscopy was used to visualize positive staining for smooth muscle actin and indicated maintenance of the smooth muscle phenotype in the BioSphincter. Additionally, BioSphincter cells were tested for the neural marker BIII-tubulin. Positive BIII-tubulin was observed by immunofluorescence, indicating that the NPC in the BioSphincters had differentiated into mature neurons.

### Example 7. Evaluation of Stability of the Bioengineered BioSphincters

To test the stability of BioSphincters at various temperature conditions for transporting Bioengineered BioSphincters, in preparation for shipping the final construct for use at clinical trial sites.

Tissue-engineered BioSphincters are currently produced at 37° C. The constructs have remained at this temperature prior to preclinical studies in animals.

For clinical use, the constructs will be transported/shipped to clinical test sites by courier. Shipping at temperatures closer to ambient room temperature as opposed to 37° C. would be easier to manage.

In this study, bioengineered BioSphincter constructs will be stored in neural Neurobasal-A Media at temperatures of 15° C., 20° C., and 25° C. for 48 hours, after which cell viability and construct functionality will be tested.

### Procedure

BioSphincters were engineered according to methods described herein above. BioSphincters were held in transportation media (Neurobasal-A Media) at 15° C., 20° C., 25° C., and 37° C. for 48 hours.

Constructs were tested for potassium chloride, acetylcholine, and electrical field stimulation (EFS) to ensure comparable functionality following CB-DVL-QCSOP-101-Physiology Analysis using Organ Bath.

A viability assay was performed to measure cell viability for each construct. Measurement of cell metabolic activity is one of the common methods to assess the viability of biomaterials. MTT (3-[4, 5-dimethylthiazol-2-yl]-2, 5 diphenyl tetrazolium bromide) dye reduction assay was carried out at different time intervals. Briefly, BioSphincters were homogenized and incubated with 0.5 mg/ml MTT solution (M5655, Sigma) at 37° C. and 5% CO₂ for 4 h. In this tenure, the MTT reagent was reduced to purple-colored formazan crystals, which were further solubilized in dimethyl sulfoxide (DMSO), and absorbance was measured at 570 nm on a microplate reader. The absorbance was considered directly proportional to the number of living cells.

### Results

### Physiological Functionality

BioSphincters incubated at different temperatures (15° C., 20° C., and 25° C.) for up to 48 hours were acclimated 1-2 hr at 37° C. and tested for physiological functionality.

BioSphincters were analyzed for the functionality of both SMC and NPC components on day 12 post-bioengineering by measuring real-time force generation on an isometric force transducer. Briefly, BioSphincters were incubated in fresh 37° C. HEPES buffer to establish a baseline for basal tone and then treated with 60-mM potassium chloride (KCl) to induce depolarization of the smooth muscle membrane. BioSphincters incubated at 37° C. for up to 48 hours were used as a control. All BioSphincters contracted in response to KCl to a similar extent without significant difference. KCl-induced depolarization results in smooth muscle contraction. Smooth muscle contraction in response to membrane depolarization reflects the maintenance of functional voltage-dependent Ca²⁺ channels in the smooth muscle cells within the BioSphincters. BioSphincters were also tested for contractility in response to the major excitatory neurotransmitter in the gut, Acetylcholine (Ach). BioSphincters contracted similarly in response to exogenous Ach without significant difference.

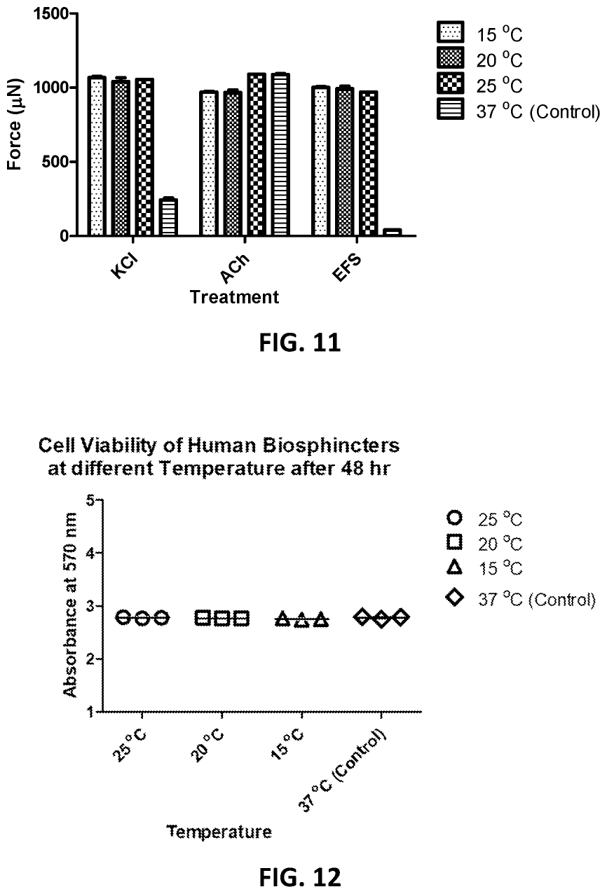

The neural component of the BioSphincter system was also tested by using electrical field stimulation (EFS). EFS (parameters: 5 Hz, 0.5 ms) was applied to the BioSphincter with parallel platinum plate electrodes in the organ bath and resulted in a relaxation response. The EFS resulted in the relaxation of the BioSphincters to a similar extent. Together, these results demonstrate that BioSphincters stored in Neurobasal-A media for up to 48 hours at 15° C., 20° C., or 25° C. maintain muscle and neural function that is comparable to control conditions. FIG. 11 shows this comparability in bar graph format; the force values mean are presented in Table 10.

TABLE 10
Summary of Physiological Functionality of BioSphincters Stored at
Different Temperatures for up to 48 Hours Compared to Control

Force ± SEM (μN)

| Storage Condition | KCl | Ach | EFS |
|---|---|---|---|
| Control 37° C. | 1072 ± 6 | 948 ± 5 | −1000 ± 21 |
| 25° C. | 1070 ± 10 | 960 ± 8 | −1008 ± 10 |
| 20° C. | 1040 ± 26 | 966 ± 17 | −993 ± 16 |
| 15° C. | 1068 ± 7 | 970 ± 6 | −1001 ± 6 |

Data showed no significant difference in BioSphincters functionality when stored at temperatures lower than 37° C. for up to 48 hours. BioSphincters performed similarly (n=3 per condition).

### Viability Assay

BioSphincters stored at 15° C., 20° C., or 25° C. for up to 48 hours were also tested for changes in cell viability. No significant changes in cell viability were observed in BioSphincters stored under these conditions when compared to control (37° C.) for up to 48 hours (FIG. 12). These results were consistent with the results obtained for physiological functionality assessment under the same conditions.

Taken together, these results indicate that BioSphincters are stable for up to 48 hours at temperatures ranging from 15° C. to 37° C.; and establish 48 hours as the point of expiry for the BioSphincter.

### Data Summary

Engineered constructs held at 15° C., 20° C. or 25° C. for 48 hours, then incubated for approximately 2 hours at 37° C. prior to testing, maintained their functional properties compared to constructs held at 37° C. through the study period.

Temperatures lower than 37° C. but higher than 15° C. for 48 hours did not appear to adversely affect cell viability, or BioSphincters construct a functionality.

### Conclusions

Tissue Engineered BioSphincter constructs can be maintained at 15° C., 20° C., and 25° C. on stainless shippers in transportation media (Neurobasal-A Media) for up to 48 hours without adversely affecting cell viability or the functionality of the construct.

Transport of bioengineered BioSphincter constructs in transportation media (Neurobasal-A Media) at temperatures ranging between 15° C. and 25° C., for up to 48 hours is therefore supported by this study.

### Example 8. Implantation of BIOSPHINCTER™ for Treatment of Severe Passive Fecal Incontinence

The present Example describes a two-center first-in-human prospective Phase I study of the immediate and long-term safety of an implanted IAS construct (BIOSPHINCTER™) bioengineered from autologous cells to treat patients with severe passive FI who have failed standard treatments.

This is a non-randomized, single group treatment study with a single arm (BioSphincters will be implanted in every study patient) and is not patient or investigator masked or blinded. After being informed of the risks, informed consent patients will undergo a biopsy procedure to collect tissue samples and will then undergo an implantation surgery of the bioengineered BIOSPHINCTER™. Patients will be followed for 36 months, post implantation. The data from this study will be analyzed to assess the safety of the treatment and the potential initial efficacy of the implanted BIOSPHINCTER™ in decreasing the number of episodes of incontinence in patients with severe FI.

### Study Procedure

Briefly, informed consent patients will enter a screening period to make sure that they meet all of the study requirements. Screening will involve: blood test for hepatitis and HIV; asking about medical history; recording personal information. (name, birth date, gender, race, ethnicity); reviewing blood test results from the last 3 months; performing a full physical exam, including a rectal exam; asking questions about the patient's condition and how it affects the patient's everyday life; performing a manometry (a test that measures pressures in anus and rectum); performing an endoscopic ultrasound (provides images of the walls of lower bowels); recording current medicines; taking a urine sample; and pregnancy test (for women of childbearing potential).

Study treatment period will start for patients who meet the study requirements. The treatment period will involve two different surgical procedures that take place about 6-8 weeks apart. Prior to both surgical procedures a number of tests and exams will be performed, including: recording blood pressure, pulse, breathing rate, temperature, and oxygen levels in blood; performing a physical exam; recording level of pain; taking a small volume (up to 20 mL or about 4 teaspoons) of blood; negative pregnancy test within 24 hours prior to surgery for women of childbearing potential; taking a urine sample; and recording current medicines and any side effects. Throughout the study, each patient will keep a bowel diary (to assess any changes to FI).

### First Surgical Procedure

The first surgery will involve biopsy procedure to collect tissue samples from small intestine and IAS in one combined operation. To take the piece of tissue from IAS, the surgeon will make a cut just at the opening of anus. An anoscope or anal retractor will be used so the surgeon can see the area from which the sample is to be taken. The internal anal sphincter will be identified under the skin or anal lining and the sample will be removed. The cut will be left open to allow drainage and prevent infection. Local anesthesia will be injected at the site of the anal cut to minimize pain afterwards. Pain relief and baths will be given to help prevent infection.

During the same combined procedure under general anesthetic, another piece of tissue from small intestine will be collected by keyhole surgery. The surgeon will make 3 or more 5-12 mm cuts in the abdomen. The abdomen will be filled with carbon dioxide to create space for the surgeon to work. The surgeon will insert a laparoscope (a thin, tube-like instrument with a light and camera) to remove a small sample from the wall of the small intestine. Once the sample is taken, the surgeon will close the intestine with stitches. The sample from the intestine will be used to grow the nerve cells in BIOSPHINCTER™ Following the biopsies, the patient will remain in the hospital for at least 23 hours for monitoring.

Site of the IAS cell harvest will be assessed postoperatively at Days 1, 7, and 21. Thus, after the first surgical procedure, the patient will return to the hospital for follow-up visits at 1 week and 3 weeks. The follow-up visits will involve: performing a physical exam; recording blood pressure, pulse, breathing rate, temperature, and oxygen levels in blood; recording pain score; taking urine sample; recording any current medicines and any side effects; pregnancy test for women of childbearing potential.

### Second Surgical Procedure

The second surgical procedure will involve implantation surgery of the BIOSPHINCTER™, and will take place 6-8 weeks after the first surgical procedure. The second surgical procedure will be performed under general anesthesia. The surgeon will make a cut (3-4 cm deep to the skin) all the way around the anus and place the BIOSPHINCTER™ in the cut. The cut will then be loosely closed with absorbable stitches. Local anesthesia will be injected around the anus to minimize pain. Pain relief and baths will be given to help prevent infection. Following the second surgical procedure, the patient will remain in the hospital for at least 23 hours for monitoring.

BIOSPHINCTER™ implantation will be assessed postoperatively at postoperative Days 1, 3, 7, 14, 28, 42 (week 6) and 56 (week 8). Additional safety assessments will be made at medical visits on Weeks 12, 24, 36 and 48-post implantation. Final safety assessments will be made at the end of Years 2 and 3. Thus, after after the second surgical procedure, the patient will return to the hospital for follow-up visits at the following timepoints: 3 days, 1 week, 2 weeks, 4 weeks, 6 weeks, 8 weeks, 12 weeks, 24 weeks, 36 weeks, 48 weeks, 2 years, 3 years. The follow-up visits will involve: performing physical exam; recording blood pressure, pulse, breathing rate, temperature, and oxygen levels in blood; recording pain score; taking urine sample; recording current medicines and any side effects; performing anal ultrasound and anal manometry; asking questions about condition and how it affects everyday life; pregnancy test for women of childbearing potential. Also, at these follow-up visits, information will be collected from medical records about FI episodes and problems or complications after the surgical procedures. Collection of such information from medical records will continue for up to 3 years after the second surgical procedure.

### Outcome Measures

### Primary Outcome Measures

1. Incidence of treatment-emergent adverse events of implanted IAS construct (BIOSPHINCTER™) for patients with severe FI who have failed standard treatment (Time Frame: 12-36 months):

To determine the safety of IAS cell harvest and the implanted BioSphincter IAS in patients with severe FI. Occurrence of adverse events.
Occurrence, severity, duration, and relationship to study procedures.
The safety of all surgical and diagnostic procedures will be assessed at inpatient hospital follow-up and outpatient clinic visits at predefined intervals.
   Biopsy: Site of IAS cell harvest will be assessed postoperatively at Days 1, 7, and 21.
   BioSphincter implantation will be assessed postoperatively at postoperative Days 1, 3, 7, 14, 28, 42 (week 6) and 56 (week 8). Additional safety assessments will be made at medical visits on Weeks 12, 24, 36 and 48-post implantation. Final safety assessments will be made at the end of Years 2 and 3.

### Secondary Outcome Measures

1. Initial efficacy of the implanted LAS construct in decreasing the number of episodes of incontinence in patients with severe FI (Time Frame: 12-48 weeks):
   Change in the number of FI episodes within a two-week period of each follow-up visit (at 12, 24, 36, and 48 weeks after implantation) as recorded in patients' bowel diaries.
2. Initial efficacy of the implanted IAS in decreasing the number of episodes of fecal urgency in patients with severe FI (Time Frame: 12-48 weeks):
   Change in the number of episodes of fecal urgency within a two-week period of each follow-up visit (at 12, 24, 36, and 48 weeks after implantation) as recorded in patients' bowel diaries.
3. Change from Baseline CCIS Score of the implanted IAS construct in improving quality of life (Time Frame: 12-48 weeks):
   Change in Cleveland Clinic Incontinence Score (CCIS) as evaluated at the time of each follow-up visit (at 12, 24, 36, and 48 weeks after implantation) and compared to baseline measures. Scored from 0 to 20 (0=complete continence, 20=complete incontinence)
4. Change from Baseline FISI Score of the implanted IAS in improving quality of life (Time Frame: 12-48 weeks):
   Change in Fecal Incontinence Severity Index (FISI) as evaluated at the time of each follow-up visit (at 12, 24, 36, and 48 weeks after implantation) and compared to baseline measures. 0 to 61, where the higher the score, the higher the perceived severity of the fecal incontinence.
5. Change from Baseline FIQOL Score of the implanted IAS in improving quality of life (Time Frame: 12-48 weeks):
   Change in Fecal Incontinence Quality of Life Scale (FIQOL) as evaluated at the time of each follow-up visit (at 12, 24, 36, and 48 weeks after implantation) and compared to baseline measures. Scales range from 1 to 5, with 1 indicating a lower functional quality-of-life status.
6. Initial efficacy of the implanted IAS by conducting Anal Rectal Manometry (ARM) measuring IASpressure to support measured clinical changes (Time Frame: 0-36 months):
   Measure of IAS pressure (mmHg) as evaluated at 12, 24, 36, and 48 weeks and at 2 and 3 years after implantation as compared with baseline measures (e.g., values from before surgery and/or values from subject with no surgery) and normal values (e.g., values from normal subject and/or healthy subject).
7. Initial efficacy of the implanted IAS by conducting Anal Rectal Manometry (ARM) measuring EAS pressure to support measured clinical changes (Time Frame: 0-36 months):
   Measure EAS pressure in mmHg as evaluated at 12, 24, 36, and 48 weeks and at 2 and 3 years after implantation as compared with baseline measures (e.g., values from before surgery and/or values from subject with no surgery) and normal values (e.g., values from normal subject and/or healthy subject).
8. Initial efficacy of the implanted IAS by conducting Anal Rectal Manometry (ARM) measuring sensation of balloon distention pressure to support measured clinical changes (Time Frame: 0-36 months):
   Measure time to sensation of balloon distention (in seconds) as evaluated at 12, 24, 36, and 48 weeks and at 2 and 3 years after implantation as compared with baseline measures (e.g., values from before surgery and/or values from subject with no surgery) and control patient normal values (e.g., values from normal subject and/or healthy subject).
9. Initial efficacy of the implanted IAS by conducting Anal Rectal Manometry (ARM) measuring presence of RAIR to support measured clinical changes (Time Frame: 0-36 months):
   Measure presence or absence of RAIR as evaluated at 12, 24, 36, and 48 weeks and at 2 and 3 years after implantation as compared with baseline measures (e.g., values from before surgery and/or values from subject with no surgery) and control patient normal values (e.g., values from normal subject and/or healthy subject).
10. Measure Physical characteristics of the Bioengineered Sphincter as assessed by endoscopic ultrasound (EUS) (Time Frame: 0-36 months):
   Measure physical characteristics of the thickness (in mm) Bioengineered Sphincter as assessed by endoscopic ultrasound at 48 weeks, 2 years and 3 years.

### Example 9. Exemplary Specifications

Exemplary in-process testing and release testing specifications (as described previously in Table 5) may include the following:
1. Endotoxin testing specification of <5 EU/kg.
2. Sterility testing for both SMCs and NPCs during drug substance testing. The sterility testing included aerobic, anaerobic, and yeast/fungal testing for the cells.
3. Rapid sterility testing of final product done at least 72 hours prior to shipment of final product.
4. Rapid mycoplasma testing of final product done at least 72 hours prior to shipment of final product.
5. MTT assay, a colorimetric assay for assessing cell metabolic activity, done as part of Potency Assay in release testing of final product.
6. Physiological functional assay done as part of Potency Assay in release testing of final product.

Exemplary results are shown in Table 11 below.

TABLE 11
Exemplary In-process and Release Testing Results

| | | | |
|---|---|---|---|
| | | | Results (n = 3 |
| Test | Method | Specifications | manufacturing runs) |

Drug Substance Testing - Cells/Spent Media
Safety

| | | | |
|---|---|---|---|
| Endotoxin | Endosafe-PTS Charles River | <5 EU/kg | 0.005 ± 0.0005 |
| | Laboratories | | |

Sterility

| | | | |
|---|---|---|---|
| Aerobic | USP <71> | Negative | Negative |
| Anaerobic | USP <71> | Negative | Negative |
| Yeast/Fungal | USP <71> | Negative | Negative |
| Gram Stain | Crystal violet stain | Negative | Negative |

Growth

| | | | |
|---|---|---|---|
| Cell Count | NucleoCounter NC-200 or | SMC: 1.25 million/ | Cell count achieved more |
| | hemacytometer count | NPC: 0.5 million for | than requirement in each |
| | | each BioSphincter | run. |
| Cell Viability | NucleoCounter NC-200 or | >70% | 89.28 ± 1.44 (SMCs) |
| | Trypan Blue exclusion | | 93.33 ± 0.80 (NPCs) |

Identity

| | | | |
|---|---|---|---|
| SMCs | BD FACSAria ™ III | αSMA (SMC) ≥80% | 98.33 ± 0.81% |
| NPCs | Immunocytochemistry | p75^{NTR} ≥80% | 99.6 ± 0.06% |

Drug Product Testing - Engineered BioSphincters (Final Product)
Rapid Sterility

| | | |
|---|---|---|
| Rapid Sterility | PCR | Negative |
| Mycoplasma | PCR | Negative |

Sterility

| | | | |
|---|---|---|---|
| Aerobic | USP <71> | Negative | Negative |
| Anaerobic | USP <71> | Negative | Negative |
| Yeast/Fungal | USP <71> | Negative | Negative |
| Gram Stain | Crystal violet stain | Negative | Negative |

Purity

| | | | |
|---|---|---|---|
| Endotoxin | Endosafe-PTS Charles River | <5 EU/kg | 0.040 ± 0.024 |
| | Laboratories | | |
| Mycoplasma | PCR | Negative | Negative |

Potency

| | | | |
|---|---|---|---|
| Physical Integrity | Visual Observation | Conforms to reference | Conforms with reference |
| Glucose/Lactate | BioProfiler Basic 2 | Metabolic detection of | Glucose uptake: 0.06 ± |
| | Chemical Analyzer | glucose uptake and | 0.007 g/BioSphincter |
| | | lactate production | Lactate Production: 0.13 ± |
| | | | 0.004 g/BioSphincter |
| MTT assay | Plate reader | >80% Viability | 97.51 ± 10.7 |
| Physiology | Mayflower Organ Bath | The generation of | Basal Tone: 261.2 ± 13 |
| Functional Assay | | Spontaneous Basal | Potassium Chloride: |
| | | Tone (250 μN ± 50 μN) | 1081.0 ± 6 |
| | | Contraction in response | Electrical Field |
| | | to Potassium Chloride | Stimulation: −959.9 ± 30 |
| | | (900 μN ± 300 μN) | |
| | | Relaxation in response | |
| | | to Electrical Field | |
| | | Stimulation | |
| | | (−900 μN ± 300 μN) | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of generating an innervated internal anal sphincter construct, comprising:
isolating smooth muscle cells (SMCs) from an anorectum tissue cell biopsy from a subject;
isolating neural progenitor cells (NPCs) from an intestinal tissue cell biopsy from the subject;
suspending the NPCs in collagen and/or laminin hydrogel, and mixing the NPCs with the SMCs to provide a cell mix;
seeding the cell mix as a single-layer hydrogel matrix on a mold and allowing gelation of the cell mix around a central post;
contacting the single-layer hydrogel matrix with a neural differentiation media; and
allowing maturation of the single-layer hydrogel matrix and differentiation of the NPCs, thereby forming the innervated internal anal sphincter construct having directionally oriented SMCs.

2. The method of claim 1, wherein the single-layer hydrogel matrix comprises NPCs and SMCs in a ratio of about 1:1 to about 1:1000.

3. The method of claim 1, wherein the single-layer hydrogel matrix comprises NPCs and SMCs in a ratio of about 1:2 to about 1:5.

4. The method of claim 1, wherein the single-layer hydrogel matrix comprises NPCs and SMCs in a ratio of about 1:2.5.

5. The method of claim 1, wherein the single-layer hydrogel matrix comprises laminin and collagen in a ratio of about 1:5 to about 1:1000.

6. The method of claim 1, wherein about 0.5×10⁶ to about 2500×10⁶ SMCs are isolated from the anorectum tissue cell biopsy from the subject.

7. The method of claim 1, further comprising obtaining the anorectum tissue cell biopsy from the subject.

8. The method of claim 7, wherein the anorectum tissue cell biopsy is obtained from internal anal sphincter tissue of the subject.

9. The method of claim 1, wherein the anorectum tissue cell biopsy weighs at least about 50 mg, preferably about 200 mg.

10. The method of claim 1, wherein about 0.2×10⁶ to about 1000×10⁶ NPCs are isolated from the intestinal tissue cell biopsy from the subject.

11. The method of claim 1, further comprising obtaining the intestinal tissue cell biopsy from the subject.

12. The method of claim 11, wherein the intestinal tissue cell biopsy is obtained from jejunal small intestine tissue of the subject.

13. The method of claim 11, wherein the intestinal tissue cell biopsy is obtained laparoscopically.

14. The method of claim 1, wherein the intestinal tissue cell biopsy weighs at least about 50 mg, preferably about 200 mg.

15. The method of claim 1, wherein the single-layer hydrogel matrix is matured to form the innervated internal anal sphincter construct in about 3-12 days.

16. The method of claim 1, wherein the innervated internal anal sphincter construct comprises less than 20% bovine collagen.

17. The method of claim 1, wherein the innervated internal anal sphincter construct comprises a central open lumen, wherein the central open lumen has a diameter of about 20-25 mm.

18. A method for treating fecal incontinence (FI) in a subject in need thereof, the method comprising:
isolating smooth muscle cells (SMCs) from an anorectum tissue cell biopsy obtained from the subject;
isolating neural progenitor cells (NPCs) from an intestinal tissue cell biopsy obtained from the subject;
suspending the NPCs in collagen and/or laminin hydrogel, and mixing the NPCs with the SMCs to provide a cell mix;
seeding the cell mix as a single-layer hydrogel matrix on a mold and allowing gelation of the cell mix around a central post;
contacting the single-layer hydrogel matrix with a neural differentiation media;
allowing maturation of the single-layer hydrogel matrix and differentiation of the NPCs, thereby forming an innervated internal anal sphincter construct having directionally oriented SMCs; and
implanting the innervated internal anal sphincter construct to the subject, wherein the implantation is through a circumferential dissection around the anorectum of the subject.

19. The method of claim 18, wherein the single-layer hydrogel matrix comprises NPCs and SMCs in a ratio of about 1:1 to about 1:1000.

20. The method of claim 18, wherein the single-layer hydrogel matrix comprises NPCs and SMCs in a ratio of about 1:2 to about 1:5.

21. The method of claim 18, wherein the single-layer hydrogel matrix comprises NPCs and SMCs in a ratio of about 1:2.5.

22. The method of claim 18, wherein the single-layer hydrogel matrix comprises laminin and collagen in a ratio of about 1:5 to about 1:1000.

23. The method of claim 18, wherein about 0.5×10⁶ to about 2500×10⁶ SMCs are isolated from the anorectum tissue cell biopsy from the subject.

24. The method of claim 18, further comprising obtaining the anorectum tissue cell biopsy from the subject.

25. The method of claim 24, wherein the anorectum tissue cell biopsy is obtained from internal anal sphincter tissue of the subject.

26. The method of claim 18, wherein the anorectum tissue cell biopsy weighs at least about 50 mg, preferably about 200 mg.

27. The method of claim 18, wherein about 0.2×10⁶ to about 1000×10⁶ NPCs are isolated from the intestinal tissue cell biopsy from the subject.

28. The method of claim 18, further comprising obtaining the intestinal tissue cell biopsy from the subject.

29. The method of claim 28, wherein the intestinal tissue cell biopsy is obtained from jejunal small intestine tissue of the subject.

30. The method of claim 28, wherein the intestinal tissue cell biopsy is obtained laparoscopically.

31. The method of claim 18, wherein the intestinal tissue cell biopsy weighs at least about 50 mg, preferably about 200 mg.

32. The method of claim 18, wherein the single layer hydrogel matrix is matured to form the innervated internal anal sphincter construct in about 3-12 days.

33. The method of claim 18, wherein multiple innervated internal anal sphincter constructs are generated and implanted in the subject.

34. The method of claim 18, wherein four innervated internal anal sphincter constructs are implanted in the subject.

35. The method of claim 1, wherein the subject is diagnosed with fecal incontinence, shows one or more symptoms of fecal incontinence, and/or is at a risk of developing fecal incontinence.

36. The method of claim 1, wherein the subject is a human.

37. The method of claim 18, wherein the subject is diagnosed with fecal incontinence, shows one or more symptoms of fecal incontinence, and/or is at a risk of developing fecal incontinence.

38. The method of claim 18, wherein the subject is a human.
